SEQUENCE LISTING (57 sequences)

SEQ ID NO: 1 - protein, 557 aa, synthetic construct
LSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEAAKAKLEQEKQIATAPQPDKKQENTTSEQEKPAPTELPPLVNKADETETPRETAPDQTPSATNTFRKILPKMNAVSQFFSQLMGTIQIVFAFILKIFK

SEQ ID NO: 2 - protein, 312 aa, synthetic construct
SKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEET

SEQ ID NO: 3 - protein, 319 aa, synthetic construct
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETHHHHHH

SEQ ID NO: 4 - protein, 16 aa, Homo sapiens
CPPCPAPELLGGPSVF

SEQ ID NO: 5 - protein, 15 aa, Homo sapiens
CPPCPAPPVAGPSVF

SEQ ID NO: 6 - protein, 16 aa, Homo sapiens
CPRCPAPELLGGPSVF

SEQ ID NO: 7 - protein, 16 aa, Homo sapiens
AHHAQAPEFLGGPSVF

SEQ ID NO: 8 - protein, 16 aa, Mus musculus
GSSCPAGNILGGPSVF

SEQ ID NO: 9 - protein, 16 aa, Mus musculus
CHKCPAPNLEGGPSVF

SEQ ID NO: 10 - protein, 16 aa, Mus musculus
CPPCAAPNLLGGPSVF

SEQ ID NO: 11 - protein, 13 aa, Mus musculus
PCICTVPEVSSVF

SEQ ID NO: 12 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 13 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 14 - protein, 349 aa, Streptococcus equi
MKTIAYPNKPHSLSAGLLTAIAIFSLASSNITYADDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 15 - protein, 315 aa, Streptococcus equi
DDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 16 - DNA, 1701 bp, synthetic construct
  source 1..1701: mol_type = other DNA
atgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgagcaccggtgaacaaatctggaagaagtacttcgaggaaaccgaaaaggagcgtattcgtctggaggaagaggcggataaggcgaaactggaacaggaccgtatccaaaaagaggcggaagagaaactggcgctggaaaaggcggaaaaagagcgtatccgtctggaagaggaagcggacaaagcgaagctggaacaagaccgtattcaaaaagaggcggaggaaaaactggcgctggagaaagcggagaaggaacgtatccgcctggaggaagaggcggacaaagcgaaactggaacaagatcgcattcagaaagaggcggaggagaagctggcgctggaaaaagcggagaaggagcgcattcgcctggaagaggaagcggataaagcgaagctggaacaggatcgcatccagaaagaggcggaagagaagctggcgctggagaaggcggagaaggagcgtatccgactggaggaagaggcggcgaaagctaaactggaacaagaaaaacaaattgcgaccgcgccgcagccggacaagaaacaagaaaacaccaccagcgaacaggagaaaccggcgccgaccgagctgccgccgctggttaacaaagcggatgaaaccgagaccccgcgtgagaccgcgccggaccaaaccccgagcgcgaccaacaccttccgtaagatcctgccgaaaatgaacgctgtgagccagttctttagccaactgatgggtaccatccagattgttttcgcgtttatcctgaagatttttaaaggcagcggtcatcaccaccaccaccactaa

SEQ ID NO: 17 - DNA, 918 bp, synthetic construct
  source 1..918: mol_type = other DNA
atgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgcatcaccaccaccaccactaa

SEQ ID NO: 18 - protein, 556 aa, synthetic construct
SKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEAAKAKLEQEKQIATAPQPDKKQENTTSEQEKPAPTELPPLVNKADETETPRETAPDQTPSATNTFRKILPKMNAVSQFFSQLMGTIQIVFAFILKIFK

SEQ ID NO: 19 - protein, 287 aa, synthetic construct
TLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEET

SEQ ID NO: 20 - protein, 312 aa, synthetic construct; identical to SEQ ID NO: 2

SEQ ID NO: 21 - protein, 449 aa, Homo sapiens
QVQLVQSGGGVVQPGRSLRLSCAASGFTFSRYTIHWVRQAPGKGLEWVAVMSYNGNNKHYADSVNGRFTISRNDSKNTLYLNMNSLRPEDTAVYYCARIRDTAMFFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Homo sapiens [sequence omitted]

SEQ ID NO: 23 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 24 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 15 aa, synthetic construct
EPKSSDKTHTCPPCP

SEQ ID NO: 28 - protein, 17 aa, synthetic construct
DKTHTCPPCPAPELLSS

SEQ ID NO: 29 - protein, 16 aa, synthetic construct
ALHNRYTQKSLSLSPG

SEQ ID NO: 30 - protein, 17 aa, synthetic construct
  VARIANT 6: note = X may be G or A
REEQYXSTYRVVSVLTV

SEQ ID NO: 31 - protein, 12 aa, synthetic construct
HYTQKSLSLSPG

SEQ ID NO: 32 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 34 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 36 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 37 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 39 - protein, 519 aa, synthetic construct
MTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDKTHTCPPCPAPELLSSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 519 aa, synthetic construct
MTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDKTHTCPPCPAPELLSSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 41 - protein, 544 aa, synthetic construct
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDKTHTCPPCPAPELLSSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 544 aa, synthetic construct
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDKTHTCPPCPAPELLSSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 43 - protein, 519 aa, synthetic construct
MTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDTPPPCPRCPAPELLSSPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 44 - protein, 544 aa, synthetic construct
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEPKSSDTPPPCPRCPAPELLSSPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 45 - protein, 519 aa, synthetic construct; identical to SEQ ID NO: 39

SEQ ID NO: 46 - protein, 519 aa, synthetic construct; identical to SEQ ID NO: 40

SEQ ID NO: 47 - protein, 544 aa, synthetic construct; identical to SEQ ID NO: 41

SEQ ID NO: 48 - protein, 544 aa, synthetic construct; identical to SEQ ID NO: 42

SEQ ID NO: 49 - protein, 519 aa, synthetic construct; identical to SEQ ID NO: 43

SEQ ID NO: 50 - protein, 544 aa, synthetic construct; identical to SEQ ID NO: 44

SEQ ID NO: 51 - protein, 563 aa, synthetic construct
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGAGCKPCICTVPEVSSVFIFPPKPKDVLMITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 52 - protein, 60 aa, synthetic construct
TGEQIWKKYFEETEPKSSDKTHTCPPCPAPELLSSPSVFLFPPKPKDTLMISRTPEVTCV

SEQ ID NO: 53 - protein, 5 aa, synthetic construct
EPKSC

SEQ ID NO: 54 - protein, 5 aa, synthetic construct
EPKSS

SEQ ID NO: 55 - protein, 10 aa, synthetic construct
APELLGGPSV

SEQ ID NO: 56 - protein, 10 aa, synthetic construct
APELLSSPSV

SEQ ID NO: 57 - protein, 4 aa, synthetic construct
EPKS

(19) United States
(12) Patent Application Publication
(10) Pub. No.: US 2024/0148841 A1
(43) Pub. Date: May 9, 2024

(54) COMPOSITIONS AND METHODS RELATED TO IMMUNOGLOBULIN PROTEASES AND FUSIONS THEREOF

(71) Applicant: Selecta Biosciences Inc., Watertown, MA (US)

(72) Inventors: Ning Li, Watertown, MA (US); Takashi Kei Kishimoto, Lexington, MA (US)

(21) Appl. No.: 18/448,842

(22) Filed: Aug. 11, 2023

Related U.S. Application Data
(60) Provisional application No. 63/463,942, filed on May 4, 2023.
(60) Provisional application No. 63/443,130, filed on Feb. 3, 2023.
(60) Provisional application No. 63/437,523, filed on Jan. 6, 2023.
(60) Provisional application No. 63/413,005, filed on Oct. 4, 2022.
(60) Provisional application No. 63/406,829, filed on Sep. 15, 2022.
(60) Provisional application No. 63/397,383, filed on Aug. 11, 2022.

(51) Int. Cl. A61K 38/48; A61K 38/38; C07K 14/76; C12N 9/52

(52) U.S. Cl. CPC A61K 38/482; A61K 38/38; C07K 14/76; C12N 9/52; C12Y 304/21; C07K 2317/24; C07K 2319/30

(57) ABSTRACT

Provided herein are compositions and methods related to compositions comprising an Ig protease fusion protein. Also provided herein are compositions and methods for therapeutic treatment, such as of autoimmune diseases, allergies, or other immunological disorders, or in combination with the administration of another therapeutic, with such Ig protease fusion protein.

### RELATED APPLICATIONS

This application claims the benefit of priority under 35 U.S.C. § 119(e) of U.S. Provisional Application Ser. No. 63/397,383, filed on Aug. 11, 2022; U.S. Provisional Application Ser. No. 63/406,829, filed on Sep. 15, 2022; U.S. Provisional Application Ser. No. 63/413,005, filed on Oct. 4, 2022; U.S. Provisional Application Ser. No. 63/437,523, filed on Jan. 6, 2023; U.S. Provisional Application Ser. No. 63/443,130, filed Feb. 3, 2023; and U.S. Provisional Application Ser. No. 63/463,942, filed May 4, 2023, the entire contents of each of which are incorporated herein by reference.

### REFERENCE TO AN ELECTRONIC SEQUENCE LISTING

The contents of the electronic sequence listing (S168170147US05-SEQ-JAV.xml; Size: 67,396 bytes; and Date of Creation: Aug. 11, 2023) is herein incorporated by reference in its entirety.

### FIELD OF THE INVENTION

Provided herein are compositions and methods related to immunoglobulin (Ig) proteases and fusions thereof. The Ig proteases and fusions thereof provided herein can be used to cleave Ig, IgG in some embodiments, and/or can have improved properties. Such Ig proteases and fusions thereof can be for use in methods of treatment, such as methods of treatment with another therapeutic. Such Ig proteases and fusions thereof can also be for use in methods of treatment, such as methods of treatment of autoimmune diseases, immunological disorders, transplantation and graft versus host disease (GVHD).

This invention also relates, at least in part, to doses of an Ig protease fusion protein for administration in combination with doses of synthetic nanocarriers attached to an immunosuppressant, and related compositions, that provide reduced immune responses. The invention also relates, at least in part, to the foregoing in combination with doses of a viral vector, such as for gene therapy, which can provide reduced immune responses and/or increased or durable transgene or nucleic acid material expression.

### SUMMARY OF THE INVENTION

In one aspect, a composition comprising an Ig protease fusion protein, comprising (i) an Ig protease domain and (ii) an Fc domain, such as an Fc as provided herein is provided, wherein, e.g., the N-terminal or C-terminal end of the Ig protease domain is fused to the Fc domain, and, optionally, wherein the Ig protease fusion protein has similar or increased activity relative to a naturally occurring Ig protease, such as any one provided herein, such as IdeS or IdeSORK (the wild-type enzymes, for example). Such activity can be any one as described herein. In one embodiment, the Ig protease fusion protein has an increased circulating half-life relative to a naturally occurring Ig protease, such as any one provided herein, such as IdeS or IdeSORK (the wild-type enzymes, for example).

In another aspect, a composition comprising an Ig protease fusion protein, comprising (i) an Ig protease domain and (ii) albumin is provided, wherein, e.g., the N-terminal or C-terminal end of the Ig protease domain is fused to the albumin, and, optionally, has similar or increased activity relative to a naturally occurring Ig protease, such as any one provided herein, such as IdeS or IdeSORK (the wild-type enzymes, for example). In one embodiment, the Ig protease fusion protein has an increased circulating half-life relative to a naturally occurring Ig protease, such as any one provided herein, such as IdeS or IdeSORK (the wild-type enzymes, for example).

In one embodiment of any one of the compositions or methods provided herein, the Ig protease fusion protein binds to a region of a target immunoglobulin (e.g., IgG or IgA), and wherein the Ig protease fusion protein cleaves the target immunoglobulin (e.g., IgG or IgA).

In one embodiment of any one of the compositions or methods provided herein, the Ig protease domain cleaves the target immunoglobulin (e.g., IgG or IgA) in a hinge region of the target immunoglobulin (e.g., IgG or IgA).

In one embodiment of any one of the compositions or methods provided herein, the Ig protease domain is or is from an Ig protease from a bacterial strain. In one embodiment of any one of the compositions or methods provided herein, the bacterial strain is a Streptococcal bacterial strain. In one embodiment of any one of the compositions or methods provided herein, the Streptococcal bacterial strain is Streptococcus pyogenes. In one embodiment of any one of the compositions or methods provided herein, the Streptococcal bacterial strain is Streptococcus equii. In one embodiment of any one of the compositions or methods provided herein, the Ig protease domain is or is from an Ig protease from Streptococcus krösus. In one embodiment of any one of the compositions or methods provided herein, the bacterial strain is a Mycoplasma bacterial strain.

In one embodiment of any one of the compositions or methods provided herein, the Ig protease domain is or is from IdeS protease. In one embodiment of any one of the compositions or methods provided herein, the Ig protease domain is or is from IdeZ protease. In one embodiment of any one of the compositions or methods provided herein, the Ig protease domain is or is from IdeMC protease.

In one embodiment of any one of the compositions or methods provided herein, the Ig protease domain is or is from IdeSORK protease.

In one embodiment of any one of the compositions or methods provided herein, the Ig protease domain comprises any one of the sequences of any one of the Ig proteases provided herein or a fragment thereof. The Ig protease may be wild-type or it may be a mutant version thereof.

In one embodiment of any one of the compositions or methods provided herein, the Fc domain comprises any one of the sequences of any one of the Fc molecules provided herein or a fragment thereof. In one embodiment of any one of the compositions or methods provided herein, the Fc domain may be wild-type or it may be a mutant version thereof. In one embodiment of any one of the compositions or methods provided herein, the Fc domain is of IgG1, IgG2, IgG3, or IgG4. In some embodiments, the Fc domain is of a human Ig. In one embodiment of any one of the compositions or methods provided herein, the Fc domain is of a mouse Ig. In one embodiment of any one of the compositions or methods provided herein, the Fc domain is specific for IgG. In one embodiment of any one of the compositions or methods provided herein, the Fc domain is specific for IgA.

In one embodiment of any one of the compositions or methods provided herein, the Fc domain (e.g., human Fc) comprises or further comprises a hinge region and a CH2 domain.

In one embodiment of any one of the compositions or methods provided herein, the Fc domain is a mutant Fc, such as to provide improved activity, such as any one of the improved activities as provided herein. In one embodiment of any one of the compositions or methods provided herein, the mutant Fc may be resistant to proteolysis by an Ig protease. In one embodiment of any one of the compositions or methods provided herein, the human Fc domain is mutated near the border of the hinge region and the CH2 domain. In one embodiment of any one of the compositions or methods provided herein, the Fc may have one or more modifications of the hinge region with or without any one or more of the other mutations as provided herein. In one embodiment of any one of the compositions or methods provided herein, the hinge region is shorter (e.g., 3× repeat) and more stable. In one embodimen of any one of the compositions or methods provided herein, any one of the Fc molecules are not glycosylated. In one embodiment of any one of the compositions or methods provided herein, the Fc domain has one or more of reduced or eliminated Fc effector functions, reduced or eliminated complement fixation, and/or enhanced binding to FcRn.

Any one of the Ig protease fusion proteins provided herein may have reduced aggregation, increased stability, increased expression, extended half-life, decreased half-life, decreased Fc to FcRn binding (e.g., IgG1 Fc to FcRn binding) and/or have Ig protease cleavage site(s) removed.

In one embodiment of any one of the compositions or methods provided herein, the Fc includes one or more mutations selected from: a GG-SS mutation in a hinge region (when the Fc comprises a hinge region); C220S (e.g., when the Fc comprises a hinge region); H435R; G236S; G237S; N297G replaced by L234A, L235A and/or P329A mutations (e.g., all 3 in one molecule); M428L and/or N434S mutations (e.g., both in one molecule); and deletion of a terminal lysine (e.g., at the terminus of the Fc molecule or antibody, or portion thereof, comprising the Fc molecule). The Fc domains as provided herein can have any combination of the mutations provided herein, such as the combinations represented by the exemplary molecules provided herein.

Any one of the foregoing can be as part of an antibody, such as a full-length antibody, or portion thereof, such as an antigen-binding portion thereof.

In one embodiment of any one of the compositions or methods provided herein, the Ig protease fusion protein is monomeric.

In one embodiment of any one of the compositions or methods provided herein, the Ig protease fusion protein is dimeric, such as homodimeric. For example, the molecules may be complexed such that they are in dimeric form. In one embodiment, the fusion protein may be a covalently bonded homodimer.

In one embodiment of any one of the compositions or methods provided herein, the Ig protease fusion protein is in knob-in-hole form.

Also provided are any one the specific fusion proteins provided herein that comprise any one of the sequences comprising a heavy chain, or portion thereof, provided herein and any one of the sequences comprising a light chain, or portion thereof, provided herein. Such specific fusion proteins can comprise any one of the light chain molecules provided herein in combination with any one of the heavy chain molecules provided herein. Compositions comprising the specific fusion proteins are also provided as are methods of using any one of the specific fusion proteins in any one of the methods provided herein. In one embodiment, the specific fusion proteins are any one of the specific combinations of sequences as provided herein.

In another aspect are the specific fusion proteins and compositions thereof provided herein, including those with the sequences below.

Any of the foregoing may be expressed in mammalian cells or non-mammalian cells and, accordingly, be a mammalian-expressed or non-mammalian expressed molecule, respectively. When the Ig protease fusion protein is a non-mammalian-expressed molecule, such as expressed from E. coli, the N297 is not mutated or at least not mutated to G or A, in one embodiment. When the Ig protease fusion protein is a mammalian-expressed molecule, the N297 may also be mutated, such as to G, in one embodiment.

In one aspect, a method of producing any one of the Ig protease fusion proteins provided herein is provided. Methods for producing the the Ig protease fusion proteins in mammalian cells, such as CHO cells, are also provided. It has been found, as an example, that Xork-Fc fusions can be expressed in such cells and purified successfully. Thus, any one of the Ig protease fusion proteins provided herein can be mammalian-expressed.

Methods for producing the Ig protease fusion proteins in non-mammalian cells, such as in E. coli, are also provided. It has been found, as an example, that Xork-Fc fusions can also be expressed in such cells successfully. Thus, any one of the Ig protease fusion proteins provided herein can be non-mammalian-expressed.

In another aspect are nucleic acids that encode any one of the Ig protease fusion proteins provided herein.

In another aspect a vector comprising any one of the nucleic acids provided herein is provided.

The compositions and methods may be for in vitro or in vivo purposes, such as cleaving of Ig, such as IgG or IgA. Thus, in one aspect methods of administering any one of the Ig protease fusion proteins or other compositions provided herein to a subject in need thereof, such as for the treatment of a disease or condition where Ig cleavage, reduction, elimination, etc. may be a benefit, are provided.

In another aspect, a method of administering any one of the Ig protease fusion proteins provided herein to a subject in need thereof, such as a subject that is being or will be administered a therapeutic biologic, is provided. The subject may be one that is being or will be treated with a viral vector. The subject may be one that is being or will be treated with a gene therapy.

In one embodiment of any one of the methods provided herein, the therapeutic biologic is a viral vector, such as an adeno-associated virus (AAV) (e.g., AAV8) viral vector. In one embodiment of any one of the methods provided herein, the subject has or is at risk of developing anti-viral vector antibodies, such as anti-AAV (e.g., AAV8) viral vector antibodies.

In one embodiment of any one of the methods provided herein, the Ig protease fusion protein and therapeutic biologic are administered concomitantly.

In another aspect, a method of administering any one of the Ig protease fusion proteins provided herein to a subject in need thereof, such as a subject that has an autoimmune disease or immunological disorder, is provided.

In another aspect, a method of administering any one of the Ig protease fusion proteins provided herein to a subject in need thereof, such as a subject that has had or will have a transplant, is provided.

In another aspect, a method of administering any one of the Ig protease fusion proteins provided herein to a subject in need thereof, such as a subject with GVHD, is provided.

In one embodiment of any one of the methods provided herein, the subject is a human. In one embodiment of any one of the methods provided herein, the subject is in need of therapeutic treatment. In one embodiment of any one of the methods provided herein, the subject is being or will be administered a therapeutic biologic, such as a viral vector, such as an AAV viral vector. In one embodiment of any one of the methods provided herein, the subject is being or will be administered a gene therapy. In one embodiment of any one of the methods provided herein, the subject has or is at risk of an autoimmune disease, immunological disorder or GVHD. In one embodiment of any one of the methods provided herein, the subject has or will undergo transplantation.

In one embodiment of any one of the methods or compositions provided herein, the Ig protease fusion protein is any one of the Ig protease fusion proteins provided herein.

In another aspect, a composition, such as comprising any one of the Ig proteases or Ig protease domains provided herein, as described in any one of the methods provided or any one of the Examples herein is provided.

In another aspect, a composition, such as comprising any one of the Fc molecules or Fc domains provided herein, as described in any one of the methods provided or any one of the Examples herein is provided.

In one embodiment, any one of the compositions provided herein is for administration according to any one of the methods provided.

In another aspect, any one of the compositions provided herein is for use in any one of the methods provided.

In another aspect, any one of the methods provided herein can further comprise administering synthetic nanocarriers that comprise an immunosuppressant. In one embodiment, the synthetic nanocarriers comprising an immunosuppressant can allow for redosing of the Ig protease fusion protein and/or another therapeutic biologic, such as a viral vector.

In one embodiment, the method therefore further comprises a further administration of the Ig protease fusion protein and/or another therapeutic biologic, such as a viral vector. In one embodiment, the further administration of the Ig protease fusion protein and another therapeutic biologic, such as a viral vector, occurs concomitantly.

In one embodiment, any one of the foregoing methods can comprise further administration of the synthetic nanocarriers comprising an immunosuppressant in combination with the further administration of the Ig protease fusion protein and/or another therapeutic biologic, such as a viral vector. In one embodiment of any one of the foregoing methods, the administration and/or further administration of the synthetic nanocarriers comprising an immunosuppressant occurs concomitantly with the Ig protease fusion protein and another therapeutic biologic, such as a viral vector.

In one embodiment of any one of the methods provided herein, the dosings of the viral vector and the synthetic nanocarriers attached to an immunosuppressant are or are about a month apart.

In one embodiment of any one of the methods provided herein, the dosings of the Ig protease fusion proteins occur prior to another therapeutic, such as a therapeutic biologic, such as a viral vector.

In an aspect, a method of manufacturing any one of the compositions or kits provided herein is provided. In one embodiment, the method of manufacturing comprises producing one or more doses or dosage forms of an Ig protease fusion protein and/or therapeutic biologic and/or a population of synthetic nanocarriers that are attached to an immunosuppressant. In another embodiment of any one of the methods of manufacturing provided, the step of producing one or more doses or dosage forms of a population of synthetic nanocarriers that are attached to an immunosuppressant comprises attaching the immunosuppressant to synthetic nanocarriers. In another embodiment of any one of the methods of manufacturing provided, the method further comprises combining the one or more doses or dosage forms of the Ig protease fusion protein and/or therapeutic biologic and/or a population of synthetic nanocarriers that are attached to an immunosuppressant in a kit.

In another aspect, any one of the compositions or kits provided herein are provided for use in any one of the methods provided herein.

### BRIEF DESCRIPTION OF THE FIGURES

FIG. 1 depicts a schematic of human IgG cleaved by IdeS or IdeZ proteases. IgG F(ab′)2 and Fc domains are shown after cleavage by IdeS or IdeZ proteases.

FIG. 2 depicts a fusion protein designed by fusing the C-terminal end of IdeS (shown normal text) with the N-terminal end of the mouse IgG1 Fc (shown in underlined text). The signal sequence is shown in bold text. The sequence corresponds to SEQ ID NO: 51.

FIGS. 3A-3B depict analyses of purified IdeS-Fc fusion protein. FIG. 3A depicts the IdeS-Fc fusion protein as a disulfide-linked homodimer of ˜120,000 daltons in non-reduced conditions and a single ˜60,000 daltons band under reducing conditions at increasing concentration of IdeS-Fc fusion protein. FIG. 3B depicts a graph of a native SEC-HPLC analysis of purified IdeS-Fc fusion protein.

FIGS. 4A-4C depicts the activity of IdeS-Fc fusion protein in vivo in rabbits. FIG. 4A is a table describing the four different treatment groups of rabbits. Group 1 rabbits were left untreated; Group 2 rabbits were immunized with 1×10¹² vector genomes/kg of AAV8 (adeno-associated virus-8) on day 1 and then not treated; Group 3 rabbits immunized with 1×10¹² vector genomes/kg of AAV8 (adeno-associated virus-8) on day 1 and were then treated with 0.5 mg of IdeS-Fc fusion protein on day 29; Group 4 rabbits immunized with 1×10¹² vector genomes/kg of AAV8 (adeno-associated virus-8) on day 1 and were then treated with 5.0 mg of IdeS-Fc fusion protein on day 29. FIG. 4B is a graph depicting total rabbit IgG titer in each treatment group measured at day 1, day 29, day 31, day 33, day 36, day 43, and day 57. FIG. 4C is a graph depicting anti-AAV8 IgG EC50 at day 1, day 29, day 31, day 33, and day 36 after dosing of AAV8 on Day 1 and Ides-Fc on Day 29.

FIGS. 5A-5B depicts amino acid sequences. FIG. 5A shows IdeSORK full length sequence of protease isolated form Streptococcus krosus (SEQ ID NO: 1); N-terminal fragment of IdeSORK having immunoglobulin protease activity (SEQ ID NO: 2); IdeSORK2.0 protein of SEQ ID NO: 2 engineered to include an additional N-terminal methionine and a C-terminal protein purification tag-His6 (SEQ ID NO: 3); SEQ ID NOs: 4-11 are the sequences of the hinge/CH2 regions of various human and mouse IgG subclasses; Full length IdeS also disclosed as NCBI Reference sequence no. WP_010922160.1 (SEQ ID NO: 12). FIG. 5B shows mature IdeS also disclosed as NCBI Reference sequence no. ADF13949.1 (SEQ ID NO: 13); Full length IdeZ also disclosed as NCBI Reference sequence no. WP_014622780.1 (SEQ ID NO: 14); Mature IdeZ (SEQ ID NO: 15); An exemplary nucleotide sequence encoding the polypeptide of SEQ ID NO: 1 engineered for expression with an N terminal histidine and a C terminal His6 (SEQ ID NO: 16); An exemplary nucleotide sequence encoding the polypeptide of SEQ ID NO: 2 engineered for expression with an N terminal histidine and a C terminal His6 (SEQ ID NO: 17). SEQ ID NO: 17 encodes SEQ ID NO: 3.

FIG. 6 depicts a schematic of IdeSORK-Fc fusion homodimer design, example sequence, and specifics. From top to bottom and left to right, SEQ ID NOs: 52, 47, and 53-57 are shown.

FIG. 7 depicts HPLC and SDS-PAGE data from IdeSORK-Fc fusion produced from CHO cells.

FIG. 8 depicts IdeSORK-Fc fusion can successfully expressed in E. coli with good solubility.

FIG. 9 is a schematic depicting engineering strategies of an IgG protease for half-life extension. The half-life of IdeSORK IgG protease can be extended by creating a human serum albumin fusion, a monomeric Fc fusion, or a homodimeric Fc fusion with IdeSORK IgG protease.

FIG. 10 is a graph depicting IgG protease-Fc fusion proteins enable AAV transduction in the presence of neutralizing human anti-AAV antibodies. Immunodeficient mice were injected with human serum on Day −3, an IgG protease on Day −2, and an AAV8 secreted alkaline phosphatase (SEAP) on Day 0. SEAP expression in relative luminance units (RLU) was measured from immune serum from mice on Day 12. Treatment groups were immune serum from mice receiving IdeS protease, IdeSORK protease, IdeSORK -HSA, or IdeSORK-Fc [KIM]. Mice were dosed at molar equivalents of IgG protease based on 1 mg/kg of native IdeS and IdeSORK.

FIG. 11 is a graph depicting IgG protease-Fc having superior activity relative to IdeS at higher doses of human serum. SEAP expression in relative luminance units (RLU) was measured from human immune serum with specific proteases. Treatment groups were human serum with IdeS, human immune serum with IdeSORK, and human immune serum with IdeSORK-Fc KIH. Treatment groups were dosed at molar equivalents of IgG protease based on 1 mg/kg of native IdeS and IdeSORK.

FIG. 12 is a graph depicting both IgG protease-Fc fusion and monomeric-Fc fusion proteins enable AAV transduction in the presence of neutralizing human anti-AAV antibodies. Immunodeficient mice were injected with human serum on Day −3, an IgG protease on Day −2, and an AAV secreted alkaline phosphatase (SEAP) on Day 0. SEAP expression in relative luminance units (RLU) was measured from immune serum from mice on Day 12. Treatment groups were immune serum from mice receiving IdeSORK, IdeSORK-Fc monomer, and IdeSORK-Fc homodimer. Mice were dosed at molar equivalents of IgG protease based on 1 mg/kg of native IdeSORK.

FIG. 13 depicts a general scheme of in vivo testing of IdeSORK protease activity against human IgG. Numbers shown correspond to days with AAV-SEAP inoculation time designated as day 0.

FIG. 14 demonstrates that Xork1.1 molecules show superior human IgG cleavage in vivo than Xork 1.0 and Xork1.2 with Xork1.1-hIgGFc-KIH restoring AAV transduction efficiency in passively immunized mice to the same levels as IdeS. SEAP activity in groups treated as indicated is shown.

FIGS. 15A-15B demonstrate total IgG levels in passively immunized mice prior to (day -2) and 48 hours after protease administration (day 0). Grouped (FIG. 15A) and individual (FIG. 15B) values are shown. The extent of decrease in human IgG levels in groups immunized with 5% human serum and treated either with Xork1.1 or IdeS is shown in FIG. 15A.

FIG. 16 demonstrates that multiple Xork1.1 and Xork1.3 molecules cleave human IgG in vivo. Xork1.1-Fc-HD, Xork1.1-Fc-HD-H435R and Xork1.3-Fc-HD enable efficient AAV transduction in passively immunized mice at standard and decreased (0.5x) doses, Xork1.3-Fc-HD-H435R is slightly less efficient at decreased dose. SEAP activity in groups treated as indicated is shown.

FIG. 17 demonstrates that Xork1.1-hIgGFc-GGSS molecule manufactured in E. coli and CHO cells is equally efficient over a wide dose range and may be superior to Xork1.1-IgGFc-H435R and Xork1.1-IgG3Fc. SEAP activity in groups treated as indicated is shown. Numbers in parentheses show SEAP activity levels in respective groups with outliers removed.

FIG. 18 demonstrates Xork1.3-hIgGFc-GGSS dimer produced in E. coli is active over a wide dose range and its activity is neither inferior to the same molecule produced in CHO cells nor to Xork1.3 H435R Fc mutant. SEAP activity in groups treated as indicated is shown. Numbers in parentheses show SEAP activity levels in respective group with outliers removed.

FIG. 19 shows the prevalence of anti-AAV IgG antibodies in humans.

FIG. 20 demonstrates the cleavage of human IgG and level of pre-existing antibodies as between IdeS and Xork.

FIGS. 21A-21C shows A) overlay of the Xork sequence on the ldeS crystal structure. Xork lgG protease has low sequence identity to ldeS but high structural similarity. B) Xork cleaves human lgG with the same specificity and mechanism as IdeS. C) Xork shows very low crossreactivity to antibodies in normal human serum compared to ldeS. Xork and ldeS crossreactivity to sera from twenty randomly healthy donors determined by ELISA.

FIGS. 22A-22E shows in vivo activity in serum passive transfer model of gene therapy A) Depiction of native Xork, monomeric Xork-Fc, and homodimeric Xork-Fc. B) Passive transfer model C) Xork 1.1-Fc has more potent in vivo activity than native Xork at equimolar amounts of enzyme dosed. D) High serum transfer. Xork 1.1 Fc monomer has more potent activity than ldeS in rescuing AAV transduction at equimolar enzyme doses in the presence of a high dose of human immune serum. E) Xork 1.3-Fc shows potent activity at 0.12 mg/kg.

FIGS. 23A-23B shows pharmacodynamics of Xork-Fc A) Xork 1.1-Fc homodimer produced in CHO cells was dosed 14 days, 7 days, 7 hours, or 15 minutes prior to or 1 day after passive transfer of human serum (Day 0). Animals were treated with AAV-SEAP 3 days after passive transfer of serum and serum SEAP activity was assessed 12 days later. B). Xork-Fc administered 7 days prior to passive transfer of human serum shows similar activity as when administered just prior serum transfer.

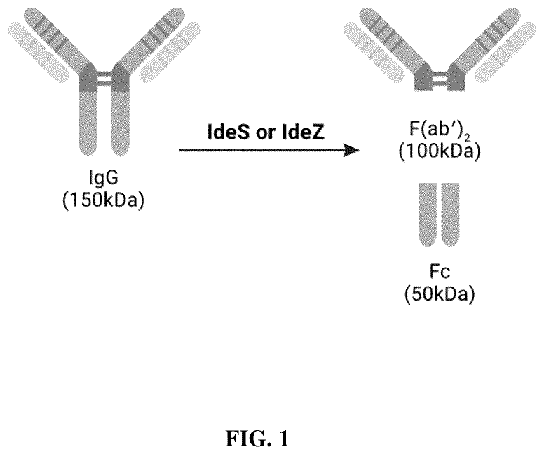

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to an Ig protease and the like. As another example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “an immunosuppressant” includes a mixture of two or more such materials or a plurality of immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

Autoimmune diseases and other immunological disorders are serious medical conditions that can be of a chronic and debilitating nature, which can lead to high medical costs and reduced quality of life. More than 80 autoimmune diseases are known, including but not limited to, type 1 diabetes, rheumatoid arthritis, systemic lupus erythematosus, and inflammatory bowel disease. Autoimmune diseases and other immunological disorders are commonly associated with a subject's immune system attacking itself and damaging its own tissues.

Immunoglobulins, which are produced by B cells and play a key role in antigen-specific host defense, can also play a pathogenic role, for example in autoimmune diseases and other immunological disorders. In normal host defense, different isotypes of secreted immunoglobulins have different roles. For example, IgG antibodies are the most abundant antibody isotype in human blood and play a major role in host defense in peripheral tissues. Autoreactive IgG antibodies can contribute to the pathogenesis of autoimmune disease. Other examples of unwanted IgG antibodies are IgG antibodies that react with donor graft tissue that can cause acute rejection of transplanted organs. For another example, IgM antibodies are typically the first secreted isotype to be produced and are involved in early host defense responses. For another example, IgA plays a critical role in host defense against mucosal pathogens. For yet another example, IgE has been implicated in the immune responses against parasites. In a pathogenic role, different types of isotypes play a pivotal role in many autoimmune diseases, such as myasthenia gravis, Grave's disease, and neuromyelitis optica. For example, IgA antibodies have been implicated in IgA nephropathy, IgA pemphigus, and linear IgA dermatosis. For another example, IgE antibodies have been implicated in allergies.

Immunoglobulins, such as of the IgG isotype, have also been associated with anti-drug antibody (ADA) responses against biologic therapies, leading to compromised efficacy or safety. Due to the primary role of immunoglobulins in host defense, various microbial pathogens have evolved proteases that selectively cleave specific immunoglobulin isotypes, or in some cases, a specific subclass of an immunoglobulin isotype, to evade the host immune response. For example, various strains of Streptococcal bacteria produce a protease (e.g., IdeS from Streptococcus pyogenes and IdeZ from Streptococcus equii) that specifically cleaves human IgG. For another example, a strain of Mycoplasma bacteria that infects dogs have evolved to produce a protease that specifically cleaves canine IgG (e.g., U.S. Patent Application No. 20190262434 A1), and a strain of Streptococcus bacteria that infects pigs have evolved to produce a protease that specifically cleaves porcine IgM (but not human IgM). In addition, several types of bacteria, including Haemophilus influenzae, Neisseria gonorrhoeae, N. meningitidis, Clostridium ramosum and Streptococcal pneumoniae, produce an IgA-specific protease. Finally, the parasite Schistosoma mansoni has been reported to produce an IgE-specific protease.

There is therapeutic potential of Ig proteases. For example, IgG proteases prevented antibody-mediated acute rejection of kidney allografts in a Phase 2 clinical trial (Jordan SC, et al. IgG Endopeptidase in Highly Sensitized Patients Undergoing Transplantation. N Engl J Med. 2017 Aug 3;377(5):442-453. doi: 10.1056/NEJMoa1612567). For another example, IgG protease has been applied to animal models of Guillain-Barre syndrome and IgG nephropathy and have been shown to enable dosing of AAV gene therapy vectors to nonhuman primates with pre-existing neutralizing antibodies against the AAV capsid. However, clinical use of microbial-derived Ig proteases are currently limited by their immunogenicity and short circulating half-life. In addition, many humans have pre-existing antibodies against Ig proteases that may compromise efficacy or safety.

Provided herein are compositions of Ig protease fusion proteins, methods of their production and methods of their use. The Ig protease fusion proteins as provided herein can be used for any purpose provided herein such as any one of the diseases, disorders or conditions provided herein. The Ig protease fusion protein can also be used in combination with the delivery of biologic therapies, including viral vector therapies. The Ig protease fusion protein can also be used in combination with the delivery of synthetic nanocarriers comprising an immunosuppressant in some embodiments.

The Ig protease fusion proteins can have improved circulating half-life in blood, such as by the inclusion of an Fc (Fc domain) or albumin. In some embodiments, the Ig protease fusion protein can have an increased half-life relative to a naturally occurring or wild-type protease, such as IdeS or IdeSORK.

Any one of the compositions described herein can be useful for the treatment of a subject as provided herein. Any one of the compositions described herein can be useful for the treatment of diseases or disorders in which Ig cleavage (e.g., IgG or IgA cleavage) can confer a benefit. It is also contemplated that the compositions described herein can be efficacious when administered in combination with other therapies. It is also contemplated that the compositions described herein can also be useful to complement other therapies, such as gene therapies or other biologic therapies.

The invention will now be described in more detail below.

### B. Definitions

“Administering” or “administration” or “administer” means giving a material to a subject in a manner such that there is a pharmacological result in the subject. This may be direct or indirect administration, such as by inducing or directing another subject, including another clinician or the subject itself, to perform the administration.

“Administration schedule” refers to administration of dosings of one or more agents according to a determined schedule. The schedule can include the number of dosings as well as the frequency of such dosings or interval between dosings. Such an administration schedule may include a number of parameters that are varied to achieve a particular objective, such as reduction of an undesired immune response to an Ig protease and/or to a therapeutic biologic (e.g., viral vector) antigen and/or increased or durable transgene or nucleic acid material expression. In embodiments, the administration schedule is any of the administration schedules as provided below in the Examples. In some embodiments, administration schedules according to the invention may be used to administer dosings to one or more test subjects. Immune responses in these test subjects can then be assessed to determine whether or not the schedule was effective in reducing an undesired immune response and/or increased or durable transgene or nucleic acid material expression. Whether or not a schedule had a desired effect can be determined using any of the methods provided herein or otherwise known in the art. For example, a sample may be obtained from a subject to which dosings provided herein have been administered according to a specific administration schedule in order to determine whether or not specific immune cells, cytokines, antibodies, etc. were reduced, generated, activated, etc. and/or specific proteins or expression products were increased, reduced or generated, etc. Useful methods for detecting the presence and/or number of immune cells include, but are not limited to, flow cytometric methods (e.g., FACS), ELISpot, proliferation responses, cytokine production, and immunohistochemistry methods. Useful methods for determining the level of protein, such as antibody, production are well known in the art and include the assays provided herein. Such assays include ELISA assays.

“Amount effective” in the context of a composition or dose for administration to a subject refers to an amount of the composition or dose that produces one or more desired responses in the subject. Therefore, in some embodiments, an amount effective is any amount of a composition or dose provided herein that produces one or more of the desired therapeutic effects and/or immune responses as provided herein. This amount can be for in vitro or in vivo purposes. For in vivo purposes, the amount can be one that a clinician would believe may have a clinical benefit for a subject in need thereof. Any one of the compositions or doses, including label doses, as provided herein can be in an amount effective.

Amounts effective can involve reducing the level of an undesired response, although in some embodiments, it involves preventing an undesired response altogether. Amounts effective can also involve delaying the occurrence of an undesired response. An amount that is effective can also be an amount that produces a desired therapeutic endpoint or a desired therapeutic result. In other embodiments, the amounts effective can involve enhancing the level of a desired response, such as a therapeutic endpoint or result. Amounts effective, can result in a therapeutic result or endpoint in any one of the subjects provided herein. The achievement of any of the foregoing can be monitored by routine methods.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

“Anti-viral vector immune response” or “immune response against a viral vector” or the like refers to any undesired immune response against a viral vector. In some embodiments, the undesired immune response is an antigen-specific immune response against the viral vector or an antigen thereof. In some embodiments, the immune response is specific to a viral antigen of the viral vector. The immune response may be an anti-viral vector antibody response, an anti-viral vector T cell immune response, such as a CD4+ T cell or CD8+ T cell immune response, or an anti-viral vector B cell immune response. Likewise, such immune responses may occur in response to other therapeutic biologics.

“Antigen” refers to a molecule that can be bound by an immunoglobulin, a B cell antigen receptor, and/or a T cell receptor. Non-limiting examples of antigens include proteins, peptides, polysaccharides, and lipopolysaccharides. Antigens can be classified as exogenous (e.g., a foreign molecule relative to a subject) or endogenous (generated, such as in cells, within a subject). Any and all types of antigens known in the art are contemplated by the present disclosure. The subject of any one of the methods provided herein can be one with an undesired immune response or undesired level of an immune response against any one of the antigens as provided herein.

“Antigen-specific” refers to any immune response that results from the presence of the antigen, or portion thereof, or that generates molecules that specifically recognize or bind the antigen. In some embodiments, when the antigen is of a viral vector, antigen-specific may mean viral vector-specific. For example, where the immune response is antigen-specific antibody production, antibodies are produced that specifically bind the antigen. As another example, where the immune response is antigen-specific B cell or CD4+ T cell proliferation and/or activity, the proliferation and/or activity results from recognition of the antigen, or portion thereof, alone or in complex with MHC molecules, B cells, etc.

“Assessing a therapeutic response” refers to any measurement or determination of the level, presence or absence, reduction in, increase in, etc. of a therapeutic response in vitro or in vivo. Such measurements or determinations may be performed on one or more samples obtained from a subject. Such assessing can be performed as a step in any one of the methods provided herein. The assessing may be assessing any one or more of the biomarkers provided herein or otherwise known in the art.

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching. In embodiments, the therapeutic biologic, synthetic nanocarriers attached to an immunosuppressant, and the Ig protease fusion protein are not attached to one another, meaning that the therapeutic biologic, synthetic nanocarriers attached to an immunosuppressant, and the Ig protease fusion protein, are not subjected to a process specifically intended to chemically associate one with another.

“Average”, as used herein, refers to the arithmetic mean unless otherwise noted.

As used herein, the term “combination therapy” is intended to define therapies which comprise the use of a combination of two or more materials/agents. Thus, references to “combination therapy”, “combinations” and the use of materials/agents “in combination” in this application may refer to materials/agents that are administered as part of the same overall treatment regimen. As such, the posology of each of the two or more materials/agents may differ: each may be administered at the same time or at different times. It will therefore be appreciated that the materials/agents of the combination may be administered sequentially (e.g., before or after) or simultaneously, either in the same pharmaceutical formulation (i.e., together), or in different pharmaceutical formulations (i.e., separately). Simultaneously in the same formulation is as a unitary formulation whereas simultaneously in different pharmaceutical formulations is non-unitary. The posologies of each of the two or more materials/agents in a combination therapy may also differ with respect to the route of administration. In one embodiment of any one of the methods provided herein, the materials/agents are administered concomitantly.

“Concomitantly” means administering two or more materials/agents to a subject in a manner that is correlated in time, preferably sufficiently correlated in time such that a first composition has an effect on a second composition, such as increasing the efficacy of the second composition, preferably the two or more materials/agents are administered in combination, so as to provide a modulation in a physiologic or immunologic response, and even more preferably the two or more materials/agents are administered in combination. In embodiments, concomitant administration may encompass administration of two or more compositions within a specified period of time. In embodiments, the two or more materials/agents are sequentially administered. In embodiments, the materials/agents may be repeatedly administered concomitantly; that is concomitant administration on more than one occasion. In any one of the embodiments of the methods or compositions provided herein, the Ig protease fusion proteins and/or synthetic nanocarriers may be administered concomitantly or repeatedly concomitantly. In some embodiments, the two or more compositions are administered within 1 month, within 1 week, within 1 day, or within 1 hour. In some embodiments, concomitant administration encompasses simultaneous administration of two or more compositions.

“Determining” or “determine” means to ascertain a factual relationship. Determining may be accomplished in a number of ways, including but not limited to performing experiments, or making projections. For instance, a dose of an immunosuppressant and/or therapeutic biologic and/or an Ig protease fusion protein, may be determined by starting with a test dose and using known scaling techniques (such as allometric or isometric scaling) to determine the dose for administration. Such may also be used to determine a protocol or administration schedule as provided herein. In another embodiment, the dose may be determined by testing various doses in a subject, i.e., through direct experimentation based on experience and guiding data. In embodiments, “determining” or “determine” comprises “causing to be determined.” “Causing to be determined” means causing, urging, encouraging, aiding, inducing or directing or acting in coordination with an entity for the entity to ascertain a factual relationship; including directly or indirectly, or expressly or impliedly.

“Dosage form” means a pharmacologically and/or immunologically active material in a medium, carrier, vehicle, or device suitable for administration to a subject. Any one of the compositions or doses provided herein may be in a dosage form.

“Dose” refers to a specific quantity of a pharmacologically and/or immunologically active material for administration to a subject for a given time.

“Dosing” means the administration of a pharmacologically and/or immunologically active material or combination of pharmacologically and/or immunologically active materials to a subject. The materials of a dosing may be administered concomitantly in any one of the methods provided herein. The materials of a dosing may be administered separately in separate compositions in any one of the methods provided herein.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier. In any one of the methods or composition provided herein, the immunosuppressant may be encapsulated in the synthetic nanocarriers.

“Expression control sequences” are any sequences that can affect expression and can include promoters, enhancers, and operators. In one embodiment of any one of the methods or compositions provided, the expression control sequence is a promoter. In one embodiment of any one of the methods or compositions provided, the expression control sequence is a liver-specific promoter or a constitutive promoter. “Liver-specific promoters” are those that exclusively or preferentially result in expression in cells of the liver. “Constitutive promoters” are those that are thought of being generally active and not exclusive or preferential to certain cells. In any one of the nucleic acids or viral vectors provided herein the promoter may be any one of the promoters provided herein.

“Fc domain” refers to the part of an Ig protease fusion protein that comprises the part of an antibody that interacts with Fc receptors or a portion thereof the interacts with a Fc receptor. In some embodiments of the disclosure, the Fc domain is of IgG1, IgG2, IgG3, or IgG4. In some embodiments, the Fc domain is of a mouse Ig. In some embodiments, the Fc domain is of a human Ig. As used herein, the term refers to a full Fc molecule or a portion thereof that interacts with Fc receptors and/or provides the necessary activitiy consistent with the disclosure and desired outcomes as provided herein.

“Generating” means causing an action, such as an immune or physiologic response (e.g., a tolerogenic immune response) to occur, either directly oneself or indirectly.

“Graft versus host disease” (GVHD) is a complication that can occur after a pluripotent cell (e.g., stem cell) or bone marrow transplant in which the newly transplanted material results in an attack on the transplant recipient's body. In some instances, GVHD takes place after a blood transfusion. Graft-versus-host-disease can be divided into acute and chronic forms. The acute or fulminant form of the disease (aGVHD) is normally observed within the first 100 days post-transplant, and is a major challenge to transplants owing to associated morbidity and mortality. The chronic form of graft-versus-host-disease (cGVHD) normally occurs after 100 days. The appearance of moderate to severe cases of cGVHD can adversely influence long-term survival.

“Homodimer Ig protease fusion protein” or “homodimeric Ig protease fusion protein” refers to the presence of two identical Ig protease domains in an Ig protease fusion protein as provided herein, which domains may be coupled or complexed to a molecule that can extend the half-life of the Ig protease domain, such as to a Fc domain. The two Ig protease domains may be coupled or complexed to each other by any means or may be coupled (such as covalently) or complexed to each other by their respective attachment to the Fc domain. The Fc domain may be comprised of units that are coupled or complexed together to form the Fc domain. Ig protease fusion proteins are referred to as “dimers” or “dimeric” when the Ig protease domains are not identical. Examples of dimeric or homodimeric Ig protease fusion proteins are provided herein.

“Identifying a subject” is any action or set of actions that allows a clinician to recognize a subject as one who may benefit from the methods or compositions provided herein or some other indicator as provided. Preferably, the identified subject is one who is in need of therapeutic treatment as provided herein. In some embodiments, the subject is identified based on symptoms (and/or lack thereof), patterns of behavior (e.g., that would put a subject at risk), and/or based on one or more tests described herein (e.g., biomarker assays). In some embodiments of any one of the methods provided herein, the subject is one that will benefit or is in need of the treatment as provided herein. In one embodiment of any one of the methods provided herein, the method further comprises identifying a subject in need of a composition or method as provided herein. The action or set of actions may be either directly oneself or indirectly, such as, but not limited to, an unrelated third party that takes an action through reliance on one's words or deeds.

“Immunoglobulin” or “Ig” refers to a glycoprotein molecule that recognizes and binds to antigens. An immunoglobulin can be classified by class or by subclass or immunoglobulin isotype. In some embodiments, an immunoglobulin of a specific class or isotype may differ in structure and/or biological function relative to another immunoglobulin of a different class or isotype.

“Immunoglobulin isotype” refers to a classification of an immunoglobulin according to the heavy chain the immunoglobulin contains (e.g., IgA contains an alpha heavy chain, IgD contains a delta heavy chain, IgE contains an epsilon heavy chain, IgG contains a gamma heavy chain, and IgM contains a mu heavy chain). In some embodiments, an immunoglobulin isotype differs in function and/or antigen response relative to a different immunoglobulin isotype. In some embodiments, immunoglobulin isotypes are further categorized by subclass (e.g., IgAl, IgA2, IgD, IgE, IgG2, IgG2a, IgG2b, IgG3, IgG4; or IgM).

“Immunoglobulin (Ig) protease” refers to an enzyme that cleaves/hydrolyzes one or more peptide bonds in an immunoglobulin. In some embodiments, a protease can be selected from a naturally occurring or wild-type or endogenous Ig proteases or variants thereof. In some embodiments, an Ig protease can be selected from an Ig protease from a bacterial strain. In some embodiments, the bacterial strain is a Streptococcal bacterial strain. In some embodiments, the bacterial strain is a Mycoplasma bacterial strain. In some embodiments, the Ig protease is IdeS protease. In some embodiments, the Ig protease is IdeZ protease. In some embodiments, the Ig protease is IdeMC protease. In some embodiments, the Ig protease is a IdeSORK protease. In some embodiments, each of the proteases herein can be wild-type or a mutant or truncated version thereof. In embodiments, the Ig protease cleaves and/or hydrolyzes one or more target immunoglobulins, such as IgG or IgA molecules.

In some embodiments, an Ig protease can be of human origin. In some embodiments, the Ig protease can comprise any portion of a human protease that can cleave the hinge region of human Ig, and such human proteases include, for example, cathepsin G and a number of matrix metalloproteinases (Ryan M H, et al. Proteolysis of purified IgGs by human and bacterial enzymes in vitro and the detection of specific proteolytic fragments of endogenous IgG in rheumatoid synovial fluid. Mol Immunol. 2008 April;45(7):1837-46. doi: 10.1016/j.molimm.2007.10.043. Epub 2007 Dec. 21. PMID: 18157932). In some embodiments, structure-based protein design can aid in the generation of protease domains that can be evaluated and optimized for specificity and/or activity.

In some embodiments, the Ig protease is a mutant version of any one of the sequences provided herein. In some embodiments, the Ig protease is a fragment of a full-length protease, such as a fragment of any one of the sequences provided herein, that possess a catalytic or hydrolytic activity of the full-length enzyme. The mutant version may comprise one or more amino acid substitutions relative to wild-type. A wide variety of Ig proteases or domains thereof can be used according to the invention in any one of the methods or compositions provided herein.

“Immunoglobulin A (IgA) protease” refers to an enzyme that cleaves and/or hydrolyzes one or more peptide bonds in an immunoglobulin A. In some embodiments, the IgA protease is from a Streptococcal bacterial strain. In some embodiments, the IgA protease is from a Neisseria bacterial strain. In some embodiments, the IgA protease is from a Clostridium bacterial strain. In some embodiments, the IgA protease is from a Capnocytophaga bacterial strain. In some embodiments, the IgA protease is from a Bacteroides bacterial strain. In some embodiments, the IgA protease is from a Gemella bacterial strain. In some embodiments, the IgA protease is from a Prevotella bacterial strain.

“Immunoglobulin G (IgG) protease” refers to an enzyme that cleaves and/or hydrolyzes one or more peptide bonds in an immunoglobulin G. In some embodiments, the IgG protease is from a Streptococcal bacterial strain. In one embodiment, the Streptococcal bacterial strain is Streptococcus pyogenes. In one embodiment, the Streptococcal bacterial strain is Streptococcus equii. In one embodiment, the Streptococcal bacterial strain is Streptococcus krosus. In some embodiments, the IgG protease is from a Mycoplasma bacterial strain. In one embodiment, the mycoplasma bacterial strain is Mycoplasma canis. In one embodiment, the IgG protease is based is any one of the IgG proteases of US Publication No. 2019-0262434 A1, the IgG proteases of which are incorporated herein by reference. In one embodiment, the IgG protease is based on a protease as described in WO2022/223818, which disclosure is incorporated herein by reference, including the Ig proteases described therein. In some embodiments, the IgG protease is a IdeSORK protease. IdeSORK may also be referred to herein as “Xork”.

“Ig protease domain” refers to the part of an Ig protease fusion protein that comprises an Ig protease or fragment or portion thereof that cleaves/hydrolyzes one or more peptide bonds in an immunoglobulin (such as in the hinge region of the immunoglobulin). In one embodiment of any one of the compositions or methods provided herein, the Ig protease domain of the Ig protease fusion protein comprises any fragment with a protease activity of any one of the Ig proteases provided herein. In some embodiments, an Ig protease domain comprises an active site of an Ig protease enzyme. In some embodiments, the Ig protease domain is or is from a Streptococcal bacterial strain. In some embodiments, the Ig protease domain is or is from an IgA protease. In some embodiments, the Ig protease domain is or is from an IgG protease. In some embodiments, the Ig protease domain is or is from IdeSORK. In some embodiments of any one of the compositions or methods provided herein, the Ig protease domain of the Ig protease fusion proteins is a full-length Ig protease. In some embodiments of any one of the compositions or methods provided herein, the Ig protease domain of the Ig protease fusion proteins is a fragment of full-length Ig protease. In some embodiments of any one of the compositions or methods provided herein, the Ig protease domain of the Ig protease fusion proteins is a mutant version of a wild-type Ig protease or fragment thereof.

“Immunosuppressant”, as used herein, means a compound that can cause a tolerogenic immune response specific to an antigen, also referred to herein as an “immunosuppressive effect”. An immunosuppressive effect generally refers to the production or expression of cytokines or other factors by an antigen-presenting cell (APC) that reduces, inhibits or prevents an undesired immune response or that promotes a desired immune response, such as a regulatory immune response, against a specific antigen. When the APC acquires an immunosuppressive function (under the immunosuppressive effect) on immune cells that recognize an antigen presented by this APC, the immunosuppressive effect is said to be specific to the presented antigen.

Immunosuppressants include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; histone deacetylase (HDAC) inhibitors, proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include methotrextate, IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. In embodiments, the immunosuppressant may comprise any of the agents provided herein.

The immunosuppressant can be a compound that directly provides the immunosuppressive effect on APCs or it can be a compound that provides the immunosuppressive effect indirectly (i.e., after being processed in some way after administration). Immunosuppressants, therefore, include prodrug forms of any of the compounds provided herein.

In embodiments of any one of the methods or compositions provided herein, the immunosuppressants provided herein are formulated with synthetic nanocarriers. In preferable embodiments, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and attached to (e.g., coupled) the one or more polymers. As another example, in one embodiment, where the synthetic nanocarrier is made up of one or more lipids, the immunosuppressant is again in addition and attached to the one or more lipids. In embodiments, such as where the material of the synthetic nanocarrier also results in an immunosuppressive effect, the immunosuppressant is an element present in addition to the material of the synthetic nanocarrier that results in an immunosuppressive effect.

“Load”, when comprised in a composition comprising a synthetic nanocarrier, such as coupled thereto, is the amount of the immunosuppressant in the composition based on the total dry recipe weight of materials in an entire synthetic nanocarrier (weight/weight). Generally, such a load is calculated as an average across a population of synthetic nanocarriers. In one embodiment, the load on average across the synthetic nanocarriers is between 0.1% and 25%, 30%, 35%, 40%, 45% or 50%. In another embodiment, the load on average across the synthetic nanocarriers is between 1% and 25%, 30%, 35%, 40%, 45% or 50%. In a further embodiment, the load is between 1% and 15%. In another embodiment, the load is between 1% and 10%. In yet a further embodiment, the load is between 5% and 15%. In still a further embodiment, the load is between 7% and 12%. In still a further embodiment, the load is between 8% and 12%. In still another embodiment, the load is between 7% and 10%. In still another embodiment, the load is between 8% and 10%. In yet a further embodiment, the load is 5%, 6%, 7%, 8%, 9%, 10%, 11%, 12%, 13%, 14%, or 15% on average across the population of synthetic nanocarriers. In any one of the methods, compositions or kits provided herein, the load of the immunosuppressant, such as rapamycin, may be any one of the loads provided herein.

The immunosuppressant (e.g., rapamycin) load of the nanocarrier in suspension can be calculated by dividing the immunosuppressant content of the nanocarrier as determined by HPLC analysis of the test article by the nanocarrier mass. The total polymer content can be measured either by gravimetric yield of the dry nanocarrier mass or by the determination of the nanocarrier solution total organic content following pharmacopeia methods and corrected for PVA content.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Preferably, a maximum dimension of at least 75%, preferably at least 80%, more preferably at least 90%, of the synthetic nanocarriers in a sample, based on the total number of synthetic nanocarriers in the sample, is less than 5 μm, 4 μm, 3 μm, 2 μm, 1 μm, 500 nm, 450 nm, 400 nm, 350 nm or 300 nm. Aspect ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1.

Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g., using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.5 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Monomer Ig protease fusion protein” or “monomeric Ig protease fusion protein” refers to the presence of one Ig protease domain in an Ig protease fusion protein as provided herein, which domain may be coupled or complexed to a molecule that can extend the half- life of the Ig protease domain, such as to a Fc domain. The Ig protease domain may be coupled or complexed to an Fc domain. The Fc domain may be comprised of units that are coupled or complexed together to form the Fc domain. In embodiments, the coupling may be covalent coupling. Examples of monomeric Ig protease fusion proteins are provided herein.

“Non-naturally occurring” or “non-natural” refers to any aspect of the present disclosure, including but not limited to polynucleotides, peptides, protein domains, proteases, and/or Ig protease fusion proteins that are modified, synthesized and/or engineered, for example, and as not being found in nature. In an embodiment of any one of the compositions or methods provided herein the Ig protease fusion protein is non-natural.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided herein may include a pharmaceutically acceptable excipient or carrier.

“Providing” means an action or set of actions that an individual performs that supply a needed item or set of items or methods for practicing of the present invention. The action or set of actions may be taken either directly oneself or indirectly.

“Providing a subject” is any action or set of actions that causes a clinician to come in contact with a subject and administer a composition provided herein thereto or to perform a method provided herein thereupon. Preferably, the subject is one who is in need of the compositions provided herein. The action or set of actions may be taken either directly oneself or indirectly. In one embodiment of any one of the methods provided herein, the method further comprises providing a subject.

“Rapalog” refers to rapamycin and molecules that are structurally related to (an analog) of rapamycin (sirolimus). Examples of rapalogs include, without limitation, temsirolimus (CCI-779), deforolimus, everolimus (RAD001), ridaforolimus (AP-23573), zotarolimus (ABT-578). Additional examples of rapalogs may be found, for example, in WO Publication WO 1998/002441 and U.S. Pat. No. 8,455,510, the disclosure of such rapalogs are incorporated herein by reference in its entirety. In any one of the methods or compositions or kits provided herein, the immunosuppressant may be a rapalog, such as rapamycin.

“Reducing immune responses” as used herein, refers to lowering or eliminating an undesired immune response against, for example, an Ig protease or other therapeutic, that would be expected to occur following administration of the Ig protease or other therapeutic (e.g., without treatment with an immunosuppressant, which can be comprised in synthetic nanocarriers). In some embodiments, the reduction in the immune response may be measured by determining an antibody titer. In some embodiments, the reduction of the immune response is an antibody titer that is durably reduced, such as for at least 1 week, 2 weeks, 1 month, 2 months, 3 months, 4 months or 5 months. In some embodiments, the subject of any one of the methods provided herein is one in need of durable antibody reduction or inhibition for at least 1 week, 2 weeks, 1 month, 2 months, 3 months, 4 months or 5 months.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. In any one of the methods or compositions provided herein, the subject is human. In any one of the methods, compositions and kits provided herein, the subject is any one of the subjects provided herein, such as one that has any one of the conditions provided herein or is in need of any one of the treatments as provided herein.

“Synthetic nanocarrier(s)” means a discrete object that is not found in nature, and that possesses at least one dimension that is less than or equal to 5 microns in size. Synthetic nanocarriers may be a variety of different shapes, including but not limited to spheroidal, cuboidal, pyramidal, oblong, cylindrical, toroidal, and the like. Synthetic nanocarriers comprise one or more surfaces.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Examples of synthetic nanocarriers include (1) the biodegradable nanoparticles disclosed in US Patent 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (7) those of Look et al., Nanogel-based delivery of mycophenolic acid ameliorates systemic lupus erythematosus in mice” J. Clinical Investigation 123(4):1741-1749(2013), (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013).

Synthetic nanocarriers may have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In an embodiment, synthetic nanocarriers that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than or equal to 1:1, 1:1.2, 1:1.5, 1:2, 1:3, 1:5, 1:7, or greater than 1:10.

A “target immunoglobulin” refers to one or more immunoglobulins cleaved by an Ig protease. In some embodiments, a target immunoglobulin may be all immunoglobulins in a specific isotype subclass (e.g., all IgG isotype subclasses, all IgA isotype subclasses, IgE, or IgD). In some embodiments, a target immunoglobulin may be a specific immunoglobulin isotype (e.g., IgA1, IgA2, IgD, IgE, IgG2, IgG2a, IgG2b, IgG3, IgG4; or IgM).

A “therapeutic biologic” refers to any protein, carbohydrate, lipid or nucleic acid that may be administered to a subject and have a therapeutic effect. In some embodiments of any one of the methods or compositions provided herein, the therapeutic biologic may be a therapeutic polynucleotide or therapeutic protein.

“Therapeutic polynucleotide” means any polynucleotide or polynucleotide-based therapy that may be administered to a subject and have a therapeutic effect. Such therapies include gene therapy, gene silencing, etc. Examples of such therapy are known in the art, and include, but are not limited to, naked RNA (including messenger RNA, modified messenger RNA, and forms of RNAi). In one embodiment of any one of the compositions or methods provided herein, the therapeutic polynucleotide is a viral vector.

“Therapeutic protein” means any protein or protein-based therapy that may be administered to a subject and have a therapeutic effect. Such therapies include protein replacement and protein supplementation therapies. Such therapies also include the administration of exogenous or foreign proteins, antibody therapies, etc. Therapeutic proteins comprise, but are not limited to, enzymes, enzyme cofactors, hormones, blood clotting factors, cytokines, growth factors, monoclonal antibodies, antibody-drug conjugates, and polyclonal antibodies.

“Transgene or nucleic acid material expression” refers to the level of the transgene or nucleic acid material expression product of a viral vector in a subject, the transgene or nucleic acid material being delivered by the viral vector. In some embodiments, the level of expression may be determined by measuring transgene protein concentrations in various tissues or systems of interest in the subject. Alternatively, when the expression product is a nucleic acid, the level of expression may be measured by nucleic acid products. Increasing expression can be determined, for example, by measuring the amount of the expression product in a sample obtained from a subject and comparing it to a prior sample. Durability of expression may be measured by similar or other methods that would be apparent to one of ordinary skill in the art. The sample may be a tissue sample. In some embodiments, the expression product can be measured using flow cytometry.

A “transplant” refers to a biological material, such as cells, tissues and organs (in whole or in part) that can be administered to a subject. Transplants may be autografts, allografts, or xenografts of, for example, a biological material such as an organ, tissue, skin, bone, nerves, tendon, neurons, blood vessels, fat, cornea, pluripotent cells, differentiated cells (obtained or derived in vivo or in vitro), etc. In some embodiments, a transplant is formed, for example, from cartilage, bone, extracellular matrix, or collagen matrices. Transplants may also be single cells, suspensions of cells and cells in tissues and organs that can be transplanted. Transplantable cells typically have a therapeutic function, for example, a function that is lacking or diminished in a recipient subject. Some non-limiting examples of transplantable cells are β-cells, hepatocytes, hematopoietic stem cells, neuronal stem cells, neurons, glial cells, or myelinating cells. Transplantable cells can be cells that are unmodified, for example, cells obtained from a donor subject and usable in transplantation without any genetic or epigenetic modifications. In other embodiments, transplantable cells can be modified cells, for example, cells obtained from a subject having a genetic defect, in which the genetic defect has been corrected, or cells that are derived from reprogrammed cells, for example, differentiated cells derived from cells obtained from a subject.

“Transplantation” refers to the process of transferring (moving) a transplant into a recipient subject (e.g., from a donor subject, from an in vitro source (e.g., differentiated autologous or heterologous native or induced pluripotent cells)) and/or from one bodily location to another bodily location in the same subject.

“Treating” refers to the administration of one or more therapeutics with the expectation that the subject may have a resulting benefit due to the administration. In some embodiments, a subject that is expected to have the presence of unwanted Ig, such as IgG or IgA, and/or in which the cleavage of Ig, such as IgG or IgA, is desired. In some embodiments, a subject that is expected to have a disease or disorder or condition is one in which a clinician believes there is a likelihood that the subject has the disease or disorder or condition. Treating may be direct or indirect, such as by inducing or directing another subject, including another clinician or the subject itself, to treat the subject.

“Undesired immune response” refers to any undesired immune response that results from exposure to an antigen, promotes or exacerbates a disease, disorder or condition provided herein (or a symptom thereof), or is symptomatic of a disease, disorder or condition provided herein. Such immune responses generally can have a negative impact on a subject's health or can be symptomatic of a negative impact on a subject's health. Undesired immune responses may be undesired humoral immune responses, which can include antigen-specific antibody production, antigen-specific B cell proliferation and/or activity or antigen-specific CD4+ T cell proliferation and/or activity. Generally, herein, these undesired immune responses are specific to a Ig protease or a therapeutic biologic, such as a viral vector, and counteract the beneficial effects desired of administration with the agent, respectively.

“Viral vector” means a vector construct with viral components, such as capsid and/or coat proteins, that has been adapted to comprise and deliver a transgene or nucleic acid material, such as one that encodes a therapeutic, such as a therapeutic protein, which transgene or nucleic acid material may be expressed as provided herein. Viral vectors can be based on, without limitation, retroviruses (e.g., murine retrovirus, avian retrovirus, Moloney murine leukemia virus (MoMuLV), Harvey murine sarcoma virus (HaMuSV), murine mammary tumor virus (MuMTV), gibbon ape leukemia virus (GaLV) and Rous Sarcoma Virus (RSV)), lentiviruses, herpes viruses, adenoviruses, adeno-associated viruses, alphaviruses, etc. Other examples are provided elsewhere herein or are known in the art. The viral vectors may be based on natural variants, strains, or serotypes of viruses, such as any one of those provided herein. The viral vectors may also be based on viruses selected through molecular evolution. The viral vectors may also be engineered vectors, recombinant vectors, mutant vectors, or hybrid vectors. In some embodiments, the viral vector is a “chimeric viral vector”. In such embodiments, this means that the viral vector is made up of viral components that are derived from more than one virus or viral vector. An AAV vector provided herein is a viral vector based on an AAV, such as AAV8, and has viral components, such as a capsid and/or coat protein, therefrom that can package for delivery the transgene or nucleic acid material.

“Viral vector antigen” means an antigen that is associated with a viral vector (i.e., the viral vector or a fragment thereof that can generate an immune response against the viral vector (e.g., the production of anti-viral vector-specific antibodies)). Viral vector antigens may be presented for recognition by the immune system (e.g., cells of the immune system, such as presented by antigen presenting cells, including but not limited to dendritic cells, B cells or macrophages). The viral vector antigens may be presented for recognition by, for example, T cells. Such antigens may be recognized by and trigger an immune response in a T cell via presentation of an epitope of the antigen bound to a Class I or Class II major histocompatability complex molecule (MHC). Viral vector antigens generally include proteins, polypeptides, peptides, polynucleotides, etc., or are contained or expressed in, on or by cells. The viral vector antigens, in some embodiments, comprise MHC Class I-restricted epitopes and/or MHC Class II-restricted epitopes and/or B cell epitopes. In some embodiments, one or more tolerogenic immune responses specific to the viral vector results with the methods, compositions or kits provided herein.

“Weight %” or “% by weight” refers to the ratio of one weight to another weight times 100. For example, the weight% can be the ratio of the weight of one component to another times 100 or the ratio of the weight of one component to a total weight of more than one component times 100. Generally, with respect to synthetic nanocarriers, the weight % is measured as an average across a population of synthetic nanocarriers or an average across the synthetic nanocarriers in a composition or suspension.

### C. Compositions and Related Methods

Provided herein are compositions of Ig protease fusion proteins and related methods, such as useful for treating a disease or disorder, e.g., by neutralizing pathogenic immunoglobulin associated with the disease or disorder. The Ig protease fusion protein of any one of the methods and compositions provided herein can have enhanced activity, such as increased half-life and/or optimized protease activity. As is described herein, any one of the compositions and methods provided herein may reduce levels of key biomarkers of a disease or disorder or markers that monitor treatment with a therapeutic. The Ig protease fusion protein of any one of the methods and compositions provided herein can be used to reduce anti-drug antibodies or to reduce viral vector (e.g., AAV vector) neutralizing antibodies. Any one of the compositions and methods provided herein may reduce levels of key biomarkers of immune responses. The Ig protease fusion protein of any one of the methods and compositions provided herein may be administered in combination with synthetic nanocarriers comprising an immunosuppressant. The Ig protease fusion protein of any one of the methods and compositions provided herein may be redosed, such as in combination with synthetic nanocarriers comprising an immunosuppressant.

### Immunoglobulin (Ig) Proteases and Ig Protease Domains Thereof

A wide variety of Ig proteases can be used according to the invention, and any protease domain thereof can be comprised of the Ig protease fusion proteins, including mutant and truncated forms, as provided herein. In some embodiments of the present disclosure, a protease domain can be selected from a naturally occurring or endogenous Ig protease or variant thereof. In some embodiments of the present disclosure, an Ig protease can be from an Ig protease from a bacterial strain. In some embodiments, the bacterial strain is a Streptococcal bacterial strain. In some embodiments, the bacterial strain is a Mycoplasma bacterial strain. In some embodiments, the bacterial strain is a Neisseria bacterial strain. In some embodiments, the bacterial strain is a Clostridium bacterial strain. In some embodiments, the bacterial strain is a Capnocytophaga bacterial strain. In some embodiments, the bacterial strain is a Bacteroides bacterial strain. In some embodiments, the bacterial strain is a Gemella bacterial strain. In some embodiments, the bacterial strain is a Prevotella bacterial strain.

In some embodiments, the Ig protease may have specificity to one or more target immunoglobulins, such as IgG or IgA immunoglobulins. In some embodiments, a target IgA may be all IgA isotype subclasses. In some embodiments, a target IgG may be all IgG isotype subclasses. In some embodiments, a target IgG may be a specific subset of the IgG isotype subclasses (e.g., IgG1, IgG2, IgG2a, IgG2b, IgG3, or IgG4). In some embodiments, the target IgG is specific to multiple (i.e., more than one) IgG subclasses or all IgG subclasses. In some embodiments, the target IgG is specific to all IgG subclasses containing lambda light chains or all IgG subclasses containing kappa light chains. In some embodiments, a target IgG may be all IgG isotype subclasses.

In some embodiments, an Ig protease can be of human origin, for example, to minimize immunogenicity. In some embodiments, the Ig protease domain can comprise any portion of a human protease that can cleave the hinge region of human Ig, and such human proteases include, for example, cathepsin G and a number of matrix metalloproteinases (Ryan MH, et al. Proteolysis of purified IgGs by human and bacterial enzymes in vitro and the detection of specific proteolytic fragments of endogenous IgG in rheumatoid synovial fluid. Mol Immunol. 2008 April;45(7):1837-46. doi: 10.1016/j.molimm.2007.10.043. Epub 2007 Dec 21. PMID: 18157932). In some embodiments, structure-based protein design can aid in the generation of protease domains that can be evaluated and optimized for specificity and/or activity. In some embodiments of any one of the compositions or methods provided herein, the Ig protease domain can be replaced by homologous or structurally related domains with similar or novel specificity, such as with sequences based on human proteins to reduce immunogenicity.

In some embodiments, the Ig protease is IdeS protease. In some embodiments, the Ig protease is IdeZ protease. In some embodiments, the Ig protease is IdeMC protease. In one embodiment, the Ig protease is any one of the IgG proteases of US Publication No. 2019-0262434 A1, the IgG proteases of which are incorporated herein by reference. In some embodiments, the Ig protease is IdeSORK protease. Exemplary sequences for such a protease are provided below.

In an embodiment of any one of the compositions or methods provided herein, the Ig protease of the present invention comprises a sequence that is at least 90%, at least 95%, at least 97%, at least 98%, or at least 99%, including all values in between, identical to any one of the sequences provided herein. Such non-identical or mutated versions include truncated or other mutant versions of any one of the Ig proteases provided herein. In an embodiment of any one of the compositions or methods provided herein, the Ig protease domain is of any one of the truncated or mutant versions if an Ig protease provided herein. Preferably, any one of the Ig proteases provided herein also exhibits an activity, such as cleaving activity against an Ig that is at least 50%, 55%, 60%, 65%, 70%, 75%, 80%, 85%, 90%, 95%, 96%, 97%, 98%, 99%, 100% or more as that of an Ig protease as provided herein.

In some embodiments, the Ig protease comprises one or more amino acid substitutions relative to wild type IdeSORK (SEQ ID NO: 1) and/or is a truncated version thereof. In one embodiment, the Ig protease comprises, consists essentially of, or consists of the amino acid sequence of SEQ ID NO: 1; the amino acid sequence of SEQ ID NO: 2; an amino acid sequence which is an N terminal fragment of the sequence of SEQ ID NO: 1; or an amino acid sequence which is at least 50% identical to the amino acid sequence of any one of the foregoing. In one embodiment, the Ig protease comprises or consists of the amino acid sequence of SEQ ID NO: 3.

In one embodiment, the Ig protease comprises a truncated form (N-terminally truncated) of a polypeptide with a sequence shown in SEQ ID NO: 18 or SEQ ID NO: 19. For example, the sequence of SEQ ID NO: 19 consists of:

TLWADGVQVDDKDFKPSTENFGTNYLAAEYGIGKGYYDINKKFDGTDDDLCSGVV AANQLHWWLDRNKDYIEKYRQQS KDNGVTIGNTDIFELNKLHDEDQSNFFDFIKKSF GNKFLQPERLLNMYINGYGYLTSQDKAKTSQPSPSKLNFFQKVFKNNLLTDKTPINDI DEFSSQTKNALQNHKVLAVSFASIKNRGLGHVVTVWGADFDENGKVVALYVTDSD DRSKNIGNAKLGMKKLRIEVSAQDSSTIKLTGFEDKNSGGSLRHLYSLSTGEQIWKK YFEET but without the N-terminal 25 amino acids and His tag of SEQ ID NO: 3.

Therefore, Ig protease may comprise the sequence of SEQ ID NO: 18 or SEQ ID NO: 20 but without at least one of amino acids 1-25 at the N-terminus thereof. In one embodiment the Ig protease comprises the sequence of SEQ ID NO: 18 or SEQ ID NO: 20 but without amino acids 1 and 2 of the N-terminus thereof. In another embodiment the Ig protease comprises the sequence of SEQ ID NO: 18 or SEQ ID NO: 20 but without amino acids 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24 or 1-25 of the N-terminus thereof. In one embodiment, the Ig protease comprises, consists essentially of or consists of the sequence of SEQ ID NO: 19.

SEQ ID NO: 18
SKRKLLKKIEKKDTSSVLTQKKQTKTLWADGVQVDDKDFK
PSTENFGTNYLAAEYGIGKGYYDINKKFDGTDDDLCSGVV
AANQLHWWLDRNKDYIEKYRQQSKDNGVTIGNTDIFELNK
LHDEDQSNFFDFIKKSFGNKFLQPERLLNMYINGYGYLTS
QDKAKTSQPSPSKLNFFQKVFKNNLLTDKTPINDIDEFSS
QTKNALQNHKVLAVSFASIKNRGLGHVVTVWGADFDENGK
VVALYVTDSDDRSKNIGNAKLGMKKLRIEVSAQDSSTIKL
TGFEDKNSGGSLRHLYSLSTGEQIWKKYFEETEKERIRLE
EEADKAKLEQDRIQKEAEEKLALEKAEKERIRLEEEADKA
KLEQDRIQKEAEEKLALEKAEKERIRLEEEADKAKLEQDR
IQKEAEEKLALEKAEKERIRLEEEADKAKLEQDRIQKEAE
EKLALEKAEKERIRLEEEAAKAKLEQEKQIATAPQPDKKQ
ENTTSEQEKPAPTELPPLVNKADETETPRETAPDQTPSAT
NTFRKILPKMNAVSQFFSQLMGTIQIVFAFILKIFK
SEQ ID NO: 19
TLWADGVQVDDKDFKPSTENFGTNYLAAEYGIGKGYYDIN
KKFDGTDDDLCSGVVAANQLHWWLDRNKDYIEKYRQQSKD
NGVTIGNTDIFELNKLHDEDQSNFFDFIKKSFGNKFLQPE
RLLNMYINGYGYLTSQDKAKTSQPSPSKLNFFQKVFKNNL
LTDKTPINDIDEFSSQTKNALQNHKVLAVSFASIKNRGLG
HVVTVWGADFDENGKVVALYVTDSDDRSKNIGNAKLGMKK
LRIEVSAQDSSTIKLTGFEDKNSGGSLRHLYSLSTGEQIW
KKYFEET
SEQ ID NO: 20
SKRKLLKKIEKKDTSSVLTQKKQTKTLWADGVQVDDKDFK
PSTENFGTNYLAAEYGIGKGYYDINKKFDGTDDDLCSGVV
AANQLHWWLDRNKDYIEKYRQQSKDNGVTIGNTDIFELNK
LHDEDQSNFFDFIKKSFGNKFLQPERLLNMYINGYGYLTS
QDKAKTSQPSPSKLNFFQKVFKNNLLTDKTPINDIDEFSS
QTKNALQNHKVLAVSFASIKNRGLGHVVTVWGADFDENGK
VVALYVTDSDDRSKNIGNAKLGMKKLRIEVSAQDSSTIKL
TGFEDKNSGGSLRHLYSLSTGEQIWKKYFEET

Any one of the Ig proteases or domains thereof can further comprise an N-terminal methionine and/or a His tag or other tag at the C-terminus. In one embodiment, the His tag or other tag may be coupled to the rest of the polypeptide by a linker. In one embodiment, the Ig protease or domain thereof is engineered to include an additional methionine at the N terminus and/or a protein purification or other tag at the C terminus, which tag may be joined to the C terminus by a linker.

In one embodiment, the Ig protease has protease activity against any immunoglobulin molecule comprising a CH2/hinge sequence as shown in any one of SEQ ID NOs: 4 to 8, wherein the Ig protease cleaves the said CH2/hinge sequence between the positions corresponding to positions 249 and 250 of human IgG according to the Kabat numbering system (positions 236 and 237 according to EU numbering system).

### Fc Domains and Modified Versions Thereof

Any Ig protease domain as provided herein can be, in some embodiments of any one of the compositions or methods provided, combined with any one of the Ig Fc domains or albumin proteins provided herein, and can be included as part of any of the Ig protease fusion proteins provided herein. In an embodiment, the Ig protease domain is engineered to be fused to a Fc molecule as provided herein. In some embodiments of any one of the methods or compositions provided, the Ig protease fusion proteins of the present invention have increased circulating half-life.

Any Fc domain can be used in any one of the compositions or methods provided and combined with any one of the Ig protease domains provided herein or included as part of any of the Ig protease fusion proteins provided herein. In some embodiments on the present disclosure, a domain of an Ig Fc can be selected from a full-length Ig Fc or a fragment thereof. In some embodiments of the disclosure, the Fc is of IgG1, IgG2, IgG3, or IgG4. In some embodiments, the Fc is of a human Ig. In one embodiment, the Fc further comprises a hinge region and/or a CH2 domain.

An exemplary sequence for a human IgG1 is as follows with the constant domain being underlined below:

(SEQ ID NO: 21)
QVQLVQSGGGVVQPGRSLRLSCAASGFTFSRYTIHWVRQA
PGKGLEWVAVMSYNGNNKHYADSVNGRFTISRNDSKNTLY
LNMNSLRPEDTAVYYCARIRDTAMFFAHWGQGTLVTVSSA
STKGPSVFPLAPSSKSTSGGTAALGCLVKDYFPEPVTVSW
NSGALTSGVHTFPAVLQSSGLYSLSSVVTVPSSSLGTQTY
ICNVNHKPSNTKVDKKVEPKSCDKTHTCPPCPAPELLGGP
SVFLFPPKPKDTLMISRTPEVTCVVVDVSHEDPEVKFNWY
VDGVEVHNAKTKPREEQYNSTYRVVSVLTVLHQDWLNGKE
YKCKVSNKALPAPIEKTISKAKGQPREPQVYTLPPSRDEL
TKNQVSLTCLVKGFYPSDIAVEWESNGQPENNYKTTPPVL
DSDGSFFLYSKLTVDKSRWQQGNVFSCSVMHEALHNHYTQ
KSLSLSPGK

An exemplary sequence for a human IgG1 constant domain is as follows:

(SEQ ID NO: 22)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK

An exemplary sequence for a human IgG2 constant domain is as follows:

(SEQ ID NO: 23)
ASTKGPSVFPLAPCSRSTSESTAALGCLVKDYFPEPVTVS
WNSGALTSGVHTFPAVLQSSGLYSLSSVVTVPSSNFGTQT
YTCNVDHKPSNTKVDKTVERKCCVECPPCPAPPVAGPSVF
LFPPKPKDTLMISRTPEVTCVVVDVSHEDPEVQFNWYVDG
VEVHNAKTKPREEQFNSTFRVVSVLTVVHQDWLNGKEYKC
KVSNKGLPAPIEKTISKTKGQPREPQVYTLPPSREEMTKN
QVSLTCLVKGFYPSDISVEWESNGQPENNYKTTPPMLDSD
GSFFLYSKLTVDKSRWQQGNVFSCSVMHEALHNHYTQKSL
SLSPGK

An exemplary sequence for a human IgG3 constant domain is as follows:

(SEQ ID NO: 24)
ASTKGPSVFPLAPCSRSTSGGTAALGCLVKDYFPEPVTVS
WNSGALTSGVHTFPAVLQSSGLYSLSSVVTVPSSSLGTQT
YTCNVNHKPSNTKVDKRVELKTPLGDTTHTCPRCPEPKSC
DTPPPCPRCPEPKSCDTPPPCPRCPEPKSCDTPPPCPRCP
APELLGGPSVFLFPPKPKDTLMISRTPEVTCVVVDVSHED
PEVQFKWYVDGVEVHNAKTKPREEQYNSTFRVVSVLTVLH
QDWLNGKEYKCKVSNKALPAPIEKTISKTKGQPREPQVYT
LPPSREEMTKNQVSLTCLVKGFYPSDIAVEWESSGQPENN
YNTTPPMLDSDGSFFLYSKLTVDKSRWQQGNIFSCSVMHE
ALHNRFTQKSLSLSPGK

An exemplary sequence for a human IgG4 constant domain is as follows:

(SEQ ID NO: 25)
ASTKGPSVFPLAPCSRSTSESTAALGCLVKDYFPEPVTVS
WNSGALTSGVHTFPAVLQSSGLYSLSSVVTVPSSSLGTKT
YTCNVDHKPSNTKVDKRVESKYGPPCPSCPAPEFLGGPSV
FLFPPKPKDTLMISRTPEVTCVVVDVSQEDPEVQFNWYVD
GVEVHNAKTKPREEQFNSTYRVVSVLTVLHQDWLNGKEYK
CKVSNKGLPSSIEKTISKAKGQPREPQVYTLPPSQEEMTK
NQVSLTCLVKGFYPSDIAVEWESNGQPENNYKTTPPVLDS
DGSFFLYSRLTVDKSRWQEGNVFSCSVMHEALHNHYTQKS
LSLSLGK

In one embodiment of any one of the compositions or methods provided herein, the Fc molecules or domains are mutated relative to wild-type. In an embodiment of any one of the compositions or methods provided herein, the Fc molecules or domains, including portions thereof, include one or more or all or any combination of the following mutations: a GG-SS mutation in a hinge region (when the Fc comprises a hinge region); C220S (e.g., when the Fc comprises a hinge region); H435R; G236S; G237S; N297G replaced by L234A, L235A and/or P329A mutations (e.g., all 3 in one molecule); M428L and/or N434S mutations (e.g., both in one molecule); and deletion of a terminal lysine (e.g., at the terminus of the Fc molecule or domain or antibody, or portion thereof, comprising the Fc molecule or domain). In some embodiments, the Fc may have one or more modifications of the hinge region with or without any one or more of the foregoing mutations. In one embodiment, the hinge region is shorter (e.g., 3× repeat) and more stable. In one embodiment, any one of the Fc molecules or domains are not glycosylated.

In an embodiment of any one of the compositions or methods provided herein, the Fc molecules or domains, including portions thereof, include one or more or all or any combination of the mutations in Table 1 below. In an embodiment of any one of the compositions or methods provided herein, the Fc molecules or domains, including portions thereof, comprise one or more or all or any combination of the sequences in Table 1 below.

In an embodiment of any one of the compositions or methods provided herein, the Fc molecules or domains comprise at least two of the mutations provided herein (any combination of two). In an embodiment of any one of the compositions or methods provided herein, the Fc molecules or domains comprise at least three of the mutations provided herein (any combination of three). In an embodiment of any one of the compositions or methods provided herein, the Fc molecules or domains comprise at least four of the mutations provided herein (any combination of four). In an embodiment of any one of the compositions or methods provided herein, the Fc molecules or domains comprise at least five of the mutations provided herein (any combination of five). In an embodiment of any one of the compositions or methods provided herein, the Fc molecules or domains comprise the mutations of any of the specific Fc molecules or domains of the specific examples of the same or examples of Ig protease fusion proteins provided herein.

TABLE 1

| Descrip- | | |
|---|---|---|
| tion | Sequence | Function |
| IgG1Fc | EPKSCDKTHTCPPCPAPELLGGPSV | wild type |
| | FLFPPKPKDTLMISRTPEVTCVVVD | |
| | VSHEDPEVKFNWYVDGVEVHNAKTK | |
| | PREEQYNSTYRVVSVLTVLHQDWLN | |
| | GKEYKCKVSNKALPAPIEKTISKAK | |
| | GQPREPQVYTLPPSRDELTKNQVSL | |
| | TCLVKGFYPSDIAVEWESNGQPENN | |
| | YKTTPPVLDSDGSFFLYSKLTVDKS | |
| | RWQQGNVFSCSVMHEALHNHYTQKS | |
| | LSLSPGK | |
| | (SEQ ID NO: 26) | |
| Hinge | EPKS(S)DKTHTCPPCP | Reduce |
| region | (SEQ ID NO: 27) | aggregation |
| C-S | | |
| mutation | | |
| Hinge | DKTHTCPPCPAPELL(SS) | IgG |
| region | (SEQ ID NO: 28) | protease |
| GG-SS | | cleavage |
| mutation | | resistent |
| Fc-H435R | ALHN(R)YTQKSLSLSPG | Shorter |
| mutation | (SEQ ID NO: 29) | half-life |
| Fc- | REEQY(G/A)STYRVVSVLTV | Effectorl |
| N297G/A | (SEQ ID NO: 30) | ess Fc |
| mutation | | |
| Fc removal | HYTQKSLSLSPG(-) | Better |
| of last | (SEQ ID NO: 31) | product |
| lysine | | purity |
| IgG1-Fc | CLVKGFYPSDIAVEWESNGQPENNY | Effectorl |
| L234A/L235 | KTTPPVLDSDGSFFLYSKLTVDKSR | ess Fc |
| A/P329A | WQQGNVFSCSVMHEALHNHYTQKSE | |
| | PKSCDKTHTCPPCPAPEAAGGPSVF | |
| | LFPPKPKDTLMISRTPEVTCVVVDV | |
| | SHEDPEVKFNWYVDGVEVHNAKTKP | |
| | REEQYNSTYRVVSVLTVLHQDWLNG | |
| | KEYKCKVSNKALAAPIEKTISKAKG | |
| | QPREPQVYTLPPSREEMTKNQVSLT | |
| | LSLSPGK | |
| | (SEQ ID NO: 32) | |
| IgG1-Fc | EPKSCDKTHTCPPCPAPEAAGGPSV | ess Fc |
| L234A/ | FLFPPKPKDTLMISRTPEVTCVVVD | longer |
| L235A/ | VSHEDPEVKFNWYVDGVEVHNAKTK | Effectorl |
| P329A/ | PREEQYNSTYRVVSVLTVLHQDWLN | with |
| M428L/ | GKEYKCKVSNKALAAPIEKTISKAK | half-life |
| N434S | GQPREPQVYTLPPSREEMTKNQVSL | |
| | TCLVKGFYPSDIAVEWESNGQPENN | |
| | YKTTPPVLDSDGSFFLYSKLTVDKS | |
| | RWQQGNVFSCSVLHEALHSHYTQKS | |
| | LSLSPGK | |
| | (SEQ ID NO: 33) | |
| IgG1Fc- | DKTHTCPPCPAPELLGGPSVFLFPP | shorter |
| shorter | KPKDTLMISRTPEVTCVVVDVSHED | hinge |
| hinge | PEVKFNWYVDGVEVHNAKTKPREEQ | |
| | YNSTYRVVSVLTVLHQDWLNGKEYK | |
| | CKVSNKALPAPIEKTISKAKGQPRE | |
| | PQVYTLPPSRDELTKNQVSLTCLVK | |
| | GFYPSDIAVEWESNGQPENNYKTTP | |
| | PVLDSDGSFFLYSKLTVDKSRWQQG | |
| | NVFSCSVMHEALHNHYTQKSLSLSP | |
| | GK | |
| | (SEQ ID NO: 34) | |
| IgG2Fc | ERKCCVECPPCPAPPVAGPSVFLFP | wild type |
| | PKPKDTLMISRTPEVTCVVVDVSHE | |
| | DPEVQFNWYVDGVEVHNAKTKPREE | |
| | QFNSTFRVVSVLTVVHQDWLNGKEY | |
| | KCKVSNKGLPAPIEKTISKTKGQPR | |
| | EPQVYTLPPSREEMTKNQVSLTCLV | |
| | KGFYPSDISVEWESNGQPENNYKTT | |
| | PPMLDSDGSFFLYSKLTVDKSRWQQ | |
| | GNVFSCSVMHEALHNHYTQKSLSLS | |
| | PGK | |
| | (SEQ ID NO: 35) | |
| IgG3Fc | ELKTPLGDTTHTCPRCPEPKSCDTP | wild type |
| | PPCPRCPEPKSCDTPPPCPRCPEPK | |
| | SCDTPPPCPRCPAPELLGGPSVFLF | |
| | PPKPKDTLMISRTPEVTCVVVDVSH | |
| | EDPEVQFKWYVDGVEVHNAKTKPRE | |
| | EQYNSTFRVVSVLTVLHQDWLNGKE | |
| | YKCKVSNKALPAPIEKTISKTKGQP | |
| | REPQVYTLPPSREEMTKNQVSLTCL | |
| | VKGFYPSDIAVEWESSGQPENNYNT | |
| | TPPMLDSDGSFFLYSKLTVDKSRWQ | |
| | QGNIFSCSVMHEALHNRFTQKSLSL | |
| | SPGK | |
| | (SEQ ID NO: 36) | |
| IgG4Fc | ESKYGPPCPSCPAPEFLGGPSVFLF | wild type |
| | PPKPKDTLMISRTPEVTCVVVDVSQ | |
| | EDPEVQFNWYVDGVEVHNAKTKPRE | |
| | EQFNSTYRVVSVLTVLHQDWLNGKE | |
| | YKCKVSNKGLPSSIEKTISKAKGQP | |
| | REPQVYTLPPSQEEMTKNQVSLTCL | |
| | VKGFYPSDIAVEWESNGQPENNYKT | |
| | TPPVLDSDGSFFLYSRLTVDKSRWQ | |
| | EGNVFSCSVMHEALHNHYTQKSLSL | |
| | SLGK | |
| | (SEQ ID NO: 37) | |
| IgG3Fc- | EPKSCDTPPPCPRCPAPELLGGPSV | wild type |
| shorter | FLFPPKPKDTLMISRTPEVTCVVVD | |
| hinge | VSHEDPEVQFKWYVDGVEVHNAKTK | |
| | PREEQYNSTFRVVSVLTVLHQDWLN | |
| | GKEYKCKVSNKALPAPIEKTISKTK | |
| | GQPREPQVYTLPPSREEMTKNQVSL | |
| | TCLVKGFYPSDIAVEWESSGQPENN | |
| | YNTTPPMLDSDGSFFLYSKLTVDKS | |
| | RWQQGNIFSCSVMHEALHNRFTQKS | |
| | LSLSPGK | |
| | (SEQ ID NO: 38) | |

The Fc molecules or domains, or portions thereof, can have any combination of the foregoing mutations, such as the combinations represented by the exemplary molecules provided herein. Thus, in one aspect is any one of the mutant Fc molecules or domains provided herein or a composition thereof. In an embodiment of any one of the compositions or methods provided herein, the Fc molecules or domains are any one of the Fc molecules provided in the Examples. Any one of the foregoing can be as part of an antibody, such as a full-length antibody, or portion thereof, such as an antigen-binding portion thereof.

Any one of the Fc molecules provided herein can have reduced aggregation, increased stability, increased expression, extended half-life, decreased half-life, decreased Fc to FcRn binding (e.g., IgG1 Fc to FcRn binding) and/or have Ig protease cleavage site(s) removed. Any one of the Fc molecules provided herein can have any one or more or all or combination of the activities of the same provided herein.

In any embodiment of any one of the compositions or methods provided herein, the Fc domain of the present invention comprises a sequence that is at least 90%, at least 95%, at least 97%, at least 98%, or at least 99%, including all values in between, identical to any one of the Fc sequences provided herein.

### Ig Protease Fusion Proteins

In another aspect are the specific Ig protease fusion proteins provided herein, including those with any one of the Ig protease domains and/or any one of the Fc domains provided herein. In another aspect are the specific Ig protease fusion proteins comprising the sequences provided herein, such as the below.

Protease-IgGFc (C-S, GG-SS)
(SEQ ID NO: 39)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDKTHTCPPCPAPELLSSPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNHYTQKSLSLSPG
Protease-IgGFc (C-S, GG-SS, H435R)
(SEQ ID NO: 40)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDKTHTCPPCPAPELLSSPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNRYTQKSLSLSPG
Protease-IgGFc (C-S, GG-SS)
(SEQ ID NO: 41)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDK
THTCPPCPAPELLSSPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNHYTQKSLSLSPG
Protease-IgGFc (C-S, GG-SS, H435R)
(SEQ ID NO: 42)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDK
THTCPPCPAPELLSSPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNRYTQKSLSLSPG
Protease-hIgG3Fc (short hinge, CS, GGSS)
(SEQ ID NO: 43)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDTPPPCPRCPAPELLSSPSVFLFPPKP
KDTLMISRTPEVTCVVVDVSHEDPEVQFKWYVDGVEVHNA
KTKPREEQYNSTFRVVSVLTVLHQDWLNGKEYKCKVSNKA
LPAPIEKTISKTKGQPREPQVYTLPPSREEMTKNQVSLTC
LVKGFYPSDIAVEWESSGQPENNYNTTPPMLDSDGSFFLY
SKLTVDKSRWQQGNIFSCSVMHEALHNRFTQKSLSLSPG
Protease-hIgG3Fc
(short hinge, CS, GGSS)
(SEQ ID NO: 44)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDT
PPPCPRCPAPELLSSPSVFLFPPKPKDTLMISRTPEVTCV
VVDVSHEDPEVQFKWYVDGVEVHNAKTKPREEQYNSTFRV
VSVLTVLHQDWLNGKEYKCKVSNKALPAPIEKTISKTKGQ
PREPQVYTLPPSREEMTKNQVSLTCLVKGFYPSDIAVEWE
SSGQPENNYNTTPPMLDSDGSFFLYSKLTVDKSRWQQGNI
FSCSVMHEALHNRFTQKSLSLSPG
Protease(Xork1.3)-IgGFc
(C-S, GG-SS)
(SEQ ID NO: 45)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDKTHTCPPCPAPELLSSPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNHYTQKSLSLSPG
Protease(Xork1.3)-IgGFc
(C-S, GG-SS, H435R)
(SEQ ID NO: 46)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDKTHTCPPCPAPELLSSPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNRYTQKSLSLSPG
Protease(Xork1.1)-IgGFc
(C-S, GG-SS)
(SEQ ID NO: 47)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDK
THTCPPCPAPELLSSPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNHYTQKSLSLSPG
Protease(Xork1.1)-IgGFc
(C-S, GG-SS, H435R)
(SEQ ID NO: 48)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDK
THTCPPCPAPELLSSPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNRYTQKSLSLSPG
Protease(Xork1.3)-hIgG3Fc
(short hinge, CS, GGSS)
(SEQ ID NO: 49)
MTLWADGVQVDDKDFKPSTENFGTNYLAAEYGIGKGYYDI
NKKFDGTDDDLCSGVVAANQLHWWLDRNKDYIEKYRQQSK
DNGVTIGNTDIFELNKLHDEDQSNFFDFIKKSFGNKFLQP
ERLLNMYINGYGYLTSQDKAKTSQPSPSKLNFFQKVFKNN
LLTDKTPINDIDEFSSQTKNALQNHKVLAVSFASIKNRGL
GHVVTVWGADFDENGKVVALYVTDSDDRSKNIGNAKLGMK
KLRIEVSAQDSSTIKLTGFEDKNSGGSLRHLYSLSTGEQI
WKKYFEETEPKSSDTPPPCPRCPAPELLSSPSVFLFPPKP
KDTLMISRTPEVTCVVVDVSHEDPEVQFKWYVDGVEVHNA
KTKPREEQYNSTFRVVSVLTVLHQDWLNGKEYKCKVSNKA
LPAPIEKTISKTKGQPREPQVYTLPPSREEMTKNQVSLTC
LVKGFYPSDIAVEWESSGQPENNYNTTPPMLDSDGSFFLY
SKLTVDKSRWQQGNIFSCSVMHEALHNRFTQKSLSLSPG
Protease(Xork1.1)-hIgG3Fc
(short hinge, CS, GGSS)
(SEQ ID NO: 50)
MSKRKLLKKIEKKDTSSVLTQKKQTKTLWADGVQVDDKDF
KPSTENFGTNYLAAEYGIGKGYYDINKKFDGTDDDLCSGV
VAANQLHWWLDRNKDYIEKYRQQSKDNGVTIGNTDIFELN
KLHDEDQSNFFDFIKKSFGNKFLQPERLLNMYINGYGYLT
SQDKAKTSQPSPSKLNFFQKVFKNNLLTDKTPINDIDEFS
SQTKNALQNHKVLAVSFASIKNRGLGHVVTVWGADFDENG
KVVALYVTDSDDRSKNIGNAKLGMKKLRIEVSAQDSSTIK
LTGFEDKNSGGSLRHLYSLSTGEQIWKKYFEETEPKSSDT
PPPCPRCPAPELLSSPSVFLFPPKPKDTLMISRTPEVTCV
VVDVSHEDPEVQFKWYVDGVEVHNAKTKPREEQYNSTFRV
VSVLTVLHQDWLNGKEYKCKVSNKALPAPIEKTISKTKGQ
PREPQVYTLPPSREEMTKNQVSLTCLVKGFYPSDIAVEWE
SSGQPENNYNTTPPMLDSDGSFFLYSKLTVDKSRWQQGNI
FSCSVMHEALHNRFTQKSLSLSPG

In an embodiment of any one of the compositions or methods provided herein, the Ig protease fusion protein of the present invention comprises a sequence that is at least 90%, at least 95%, at least 97%, at least 98%, or at least 99%, including all values in between, identical to any one of the Ig protease fusion protein sequences provided herein.

In one embodiment of any one of the Ig protease fusion proteins provided herein, the fusion protein is in monomeric form. In another embodiment, the fusion protein may be complexed with another fusion protein such that they are in dimeric form. In one embodiment, the fusion protein dimer is a homodimer. When a homodimer, the disulfide bond in the hinge region of the Fc may dimerize the fusion proteins. In an embodiment, the dimerization may be the result of covalent bonding.

The units to form a Fc domain main be coupled or complexed by the knob-in-hole mechanism (or KIH mechanism). “Knob-in-hole” or “KIH” refers to a strategy in antibody engineering that is used for dimerization in the production of antibodies. Amino acids that form the interface of a domain in Ig, IgG for example, can be mutated at positions that affect domain interactions to promote dimer formation. A knob is represented by an amino acid with a large side change (e.g., a tyrosine) and a hole is represented by an amino acid with a small side chain (e.g., threonine). An amino acid with a large side chain (knob) can be introduced into a heavy chain of an antibody that specifically binds to a first antigen and an amino acid with a small side chain (hole) can be introduced into a heavy chain of an antibody specifically binding to a second antigen. Co-expression of the two with the knob and hole mutations can result in the formation of a dimer due to the interaction between the heavy chain hole and the heavy chain knob. Any one of the Ig protease fusion proteins may in KIH form.

Any of the foregoing may be expressed in mammalian cells or non-mammalian cells and, accordingly, be a mammalian-expressed or non-mammalian expressed molecule, respectively. When the Fc fusion is a non-mammalian-expressed molecule, such as expressed from E. coli, the N297 is not mutated or at least not mutated to G or A, in one embodiment. When the Fc fusion is a mammalian-expressed molecule, the N297 may also be mutated, such as to G, in one embodiment.

Methods for producing the Fc molecules or fusions thereof in mammalian cells, such as CHO cells, are also provided. It has been found, as an example, that an Ig protease (Xork)-Fc fusion can be expressed in such cells and purified successfully. Thus, any one of the Fc molecules or fusions thereof provided herein can be a mammalian-expressed Fc molecule or fusion thereof. Methods for producing the Fc molecules or fusions thereof in non-mammalian cells, such as E.coli cells, are also provided. Method for such production are also known in the art (See, for example, U.S. Pat. No. 6,835,809, which methods are incorporated herein by reference). It has been found, as an example, that an Ig protease (Xork)-Fc fusion can be expressed in such cells and purified successfully. Thus, any one of the Fc molecules or fusions thereof provided herein can be a non-mammalian-expressed Fc molecule or fusion thereof.

### Albumin Proteins

Any albumin protein can be used, in some embodiments of any one of the compositions or methods provided and combined with any one of the Ig protease domains provided herein or included as part of any one of the Ig protease fusion proteins provided herein. In some embodiments, the albumin protein may be mutated, truncated or engineered to improve binding to FcRn. In some embodiments, the albumin protein is human albumin protein.

### Transgenes and Viral Vectors

The transgene or nucleic acid material, such as of the viral vectors, provided herein may encode any protein or portion thereof or nucleic acid or portion thereof beneficial to a subject, such as one with a disease or disorder. In embodiments, the subject has or is suspected of having a disease or disorder whereby the subject's endogenous version of the protein is defective or produced in limited amounts or not at all. The subject may be one with any one of the diseases or disorders as provided herein, and the transgene or nucleic acid material is one that encodes any one of the therapeutic proteins or portion thereof as provided herein. The transgene or nucleic acid material provided herein may encode a functional version of any protein that through some defect in the endogenous version of which in a subject (including a defect in the expression of the endogenous version) results in a disease or disorder in the subject.

The sequence of a transgene or nucleic acid material may also include an expression control sequence. Expression control sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include liver-specific promoter sequences and constitutive promoter sequences, such as any one that may be provided herein. Generally, promoters are operatively linked upstream (i.e., 5′) of the sequence coding for a desired expression product. The transgene also may include a suitable polyadenylation sequence operably linked downstream (i.e., 3′) of the coding sequence.

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, adeno-associated virus (AAV)-based vectors.

The viral vectors provided herein can be based on adeno-associated viruses (AAVs). AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699.

The adeno-associated virus on which a viral vector is based may be of a specific serotype, such as AAV8 or AAV2. In some embodiments of any one of the methods or compositions provided herein, therefore, the AAV vector is an AAV8 or AAV2 vector. Viral vectors can be made with methods known to those of ordinary skill in the art or as otherwise described herein. For example, viral vectors can be constructed and/or purified using the methods set forth, for example, in U.S. Pat. No. 4,797,368 and Laughlin et al., Gene, 23, 65-73 (1983).

Viral vectors, such as AAV vectors, may be produced using recombinant methods. For example, the methods can involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a viral vector in a capsid may be provided to the host cell in trans. Alternatively, any one or more of the required components (e.g., recombinant viral vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant viral vector, rep sequences, cap sequences, and helper functions for producing the viral vector may be delivered to the packaging host cell using any appropriate genetic element. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV vectors may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (such as comprising a transgene) to be packaged into AAV particles, an AAV helper function vector, and an accessory function vector. Generally, an AAV helper function vector encodes AAV helper function sequences (rep and cap), which function in trans for productive AAV replication and encapsulation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Other methods for producing viral vectors are known in the art. Moreover, viral vectors are available commercially.

### Uses of the Ig Protease Fusion Proteins

It is contemplated by the present disclosure that the Ig protease fusion proteins may be administered to a subject. In some embodiments of the present disclosure, methods of administering the Ig protease fusion proteins to a subject are provided, wherein the Ig protease fusion proteins cleave immunoglobulin, such as IgG or IgA, in a subject. In some embodiments, methods of administering the Ig protease fusion proteins to a subject are provided.

It is contemplated that any of the Ig protease fusion proteins contemplated by the present disclosure can be used for therapeutic treatment, such as of autoimmune diseases, allergies, or other immunological disorders. Any of the Ig protease fusion proteins can be used with biologic therapies, such as therapeutic protein therapies or therapeutic polynucleotide therapies, such as viral vector therapies, etc.

Administration according to the present invention may be by a variety of routes, including but not limited to subcutaneous, intravenous and intraperitoneal routes. For example, the mode of administration for the composition of any one of the treatment methods provided may be by intravenous administration. The compositions referred to herein may be manufactured and prepared for administration using conventional methods.

The compositions of the invention can be administered in effective amounts, such as the effective amounts described herein. In some embodiments of any one of the methods or compositions provided, repeated cycles of administration of the Ig protease fusion proteins are contemplated.

Aspects of the invention relate to determining a protocol for the methods of administration as provided herein. A protocol can be determined by varying at least the frequency, dosage amount of the Ig protease fusion proteins and/or synthetic nanocarriers and/or other therapeutic and subsequently assessing a desired or undesired therapeutic response or immune response. The protocol can comprise at least the frequency of the administration and doses of the Ig protease fusion proteins and/or synthetic nanocarriers and/or other therapeutic. Any one of the methods provided herein can include a step of determining a protocol or the administering steps are performed according to a protocol that was determined to achieve any one or more of the desired results as provided herein.

### Synthetic Nanocarriers

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, various elements of the synthetic nanocarriers can be attached to the polymer.

The immunosuppressants can be attached to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly((3-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(l,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. attached) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly [a-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Compositions according to the invention can comprise elements, such as immunosuppressants, in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions, such as those comprising immunosuppressants, are suspended in sterile saline solution for injection together with a preservative.

In embodiments, when preparing synthetic nanocarriers as carriers, methods for attaching components to the synthetic nanocarriers may be useful. If the component is a small molecule it may be of advantage to attach the component to a polymer prior to the assembly of the synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

In certain embodiments, the attaching can be a covalent linker. In embodiments, immunosuppressants according to the invention can be covalently attached to the external surface via a 1,2,3-triazole linker formed by the 1,3-dipolar cycloaddition reaction of azido groups on the surface of the nanocarrier with immunosuppressant containing an alkyne group or by the 1,3-dipolar cycloaddition reaction of alkynes on the surface of the nanocarrier with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S-S) bond between two sulfur atoms of the form, for instance, of R1-S-S-R2. A disulfide bond can be formed by thiol exchange of a component containing thiol/mercaptan group(-SH) with another activated thiol group on a polymer or nanocarrier or a nanocarrier containing thiol/mercaptan groups with a component containing activated thiol group.

A triazole linker, specifically a 1,2,3-triazole of the form

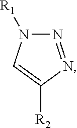

wherein R1 and R2 may be any chemical entities, is made by the 1,3-dipolar cycloaddition reaction of an azide attached to a first component such as the nanocarrier with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

A thioether linker is made by the formation of a sulfur-carbon (thioether) bond in the form, for instance, of R1-S-R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated to the nanocarrier via non-covalent conjugation methods. For example, a negative charged immunosuppressant can be conjugated to a positive charged nanocarrier through electrostatic adsorption. A component containing a metal ligand can also be conjugated to a nanocarrier containing a metal complex via a metal-ligand complex.

In embodiments, the component can be attached to a polymer, for example polylactic acid-block-polyethylene glycol, prior to the assembly of the synthetic nanocarrier or the synthetic nanocarrier can be formed with reactive or activatible groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier, or it can be attached by encapsulation during the formation of the synthetic nanocarrier.

As examples, synthetic nanocarriers comprising rapamycin can be produced or obtainable by one of the following methods:
1) PLA with an inherent viscosity of 0.41 dL/g is purchased from Evonik Industries (Rellinghauser Straβe 1-11 45128 Essen, Germany), product code Resomer Select 100 DL 4A. PLA-PEG-OMe block co-polymer with a methyl ether terminated PEG block of approximately 5,000 Da and an overall inherent viscosity of 0.50 DL/g is purchased from Evonik Industries (Rellinghauser Straβe 1-11 45128 Essen, Germany), product code Resomer Select 100 DL mPEG 5000 (15 wt % PEG). Rapamycin is purchased from Concord Biotech Limited (1482-1486 Trasad Road, Dholka 382225, Ahmedabad India), product code SIROLIMUS. EMPROVE® Polyvinyl Alcohol 4-88, USP (85-89% hydrolyzed, viscosity of 3.4-4.6 mPa·s) is purchased from MilliporeSigma (EMD Millipore, 290 Concord Road Billerica, Massachusetts 01821), product code 1.41350. Dulbecco's phosphate buffered saline 1X (DPBS) is purchased from Lonza (Muenchensteinerstrasse 38, CH-4002 Basel, Switzerland), product code 17-512Q. Sorbitan monopalmitate is purchased from Croda International (300-A Columbus Circle, Edison, NJ 08837), product code SPAN 40. Solutions are prepared as follows. Solution 1 is prepared by dissolving PLA at 150 mg/mL and PLA-PEG-Ome at 50 mg/mL in dichloromethane. Solution 2 is prepared by dissolving rapamycin at 100 mg/mL in dichloromethane. Solution 3 is prepared by dissolving SPAN 40 at 50 mg/mL in dichloromethane. Solution 4 is prepared by dissolving PVA at 75 mg/mL in 100 mM phosphate buffer pH 8. 0/W emulsions are prepared by adding Solution 1 (0.50 mL), Solution 2 (0.12 mL), Solution 3 (0.10 mL), and dichloromethane (0.28 mL), in a thick walled glass pressure tube. The combined organic phase solutions are then mixed by repeat pipetting. To this mixture, Solution 4 (3 mL), is added. The pressure tube is then vortex mixed for 10 seconds. Next, the crude emulsion is homogenized by sonication at 30% amplitude for 1 minute using a Branson Digital Sonifier 250 with a ⅛″ tapered tip, and the pressure tube immersed in an ice water bath. The emulsion is then added to a 50 mL beaker containing DPBS (30 mL). This is stirred at room temperature for 2 hours to allow the dichloromethane to evaporate and for the nanocarriers to form. A portion of the nanocarriers is washed by transferring the nanocarrier suspension to a centrifuge tube and centrifuging at 75,600× g at 4 ° C. for 50 minutes, removing the supernatant, and re-suspended the pellet in DPBS containing 0.25% w/v PVA. The wash procedure is repeated and the pellet is re-suspended in DPBS containing 0.25% w/v PVA to achieve a nanocarrier suspension having a nominal concentration of 10 mg/mL on a polymer basis. The nanocarrier suspension is then filtered using a 0.22 μm PES membrane syringe filter from MilliporeSigma (EMD Millipore, 290 Concord Rd. Billerica MA, product code SLGP033RB). The filtered nanocarrier suspension is stored at −20° C.

2) PLA with an inherent viscosity of 0.41 dL/g is purchased from Evonik Industries (Rellinghauser Straβe 1-11 45128 Essen, Germany), product code Resomer Select 100 DL 4A. PLA-PEG-OMe block co-polymer with a methyl ether terminated PEG block of approximately 5,000 Da and an overall inherent viscosity of 0.50 DL/g is purchased from Evonik Industries (Rellinghauser Straβe 1-11 45128 Essen, Germany), product code Resomer Select 100 DL mPEG 5000 (15 wt % PEG). Rapamycin is purchased from Concord Biotech Limited (1482-1486 Trasad Road, Dholka 382225, Ahmedabad India), product code SIROLIMUS. Sorbitan monopalmitate is purchased from Sigma-Aldrich (3050 Spruce St., St. Louis, MO 63103), product code 388920. EMPROVE® Polyvinyl Alcohol (PVA) 4-88, USP (85-89% hydrolyzed, viscosity of 3.4-4.6 mPa·s) is purchased from MilliporeSigma (EMD Millipore, 290 Concord Road Billerica, Massachusetts 01821), product code 1.41350. Dulbecco's phosphate buffered saline 1× (DPBS) is purchased from Lonza (Muenchensteinerstrasse 38, CH-4002 Basel, Switzerland), product code 17-512Q. Solutions are prepared as follows: Solution 1: A polymer, rapamycin, and sorbitan monopalmitate mixture is prepared by dissolving PLA at 37.5 mg/mL, PLA-PEG-Ome at 12.5 mg/mL, rapamycin at 8 mg/mL, and sorbitan monopalmitate at 2.5 in dichloromethane. Solution 2: Polyvinyl alcohol is prepared at 50 mg/mL in 100 mM pH 8 phosphate buffer. An O/W emulsion is prepared by combining Solution 1 (1.0 mL) and Solution 2 (3 mL) in a small glass pressure tube, and vortex mixed for 10 seconds. The formulation is then homogenized by sonication at 30% amplitude for 1 minute using a Branson Digital Sonifier 250 with a 1/8″ tapered tip, with the pressure tube immersed in an ice water bath. The emulsion is then added to a 50 mL beaker containing DPBS (15 mL), and covered with aluminum foil. A second O/W emulsion is prepared using the same materials and method as above and then added to the same beaker using a fresh aliquot of DPBS (15 mL). The combined emulsion is then left uncovered and stirred at room temperature for 2 hours to allow the dichloromethane to evaporate and for the nanocarriers to form. A portion of the nanocarriers is washed by transferring the nanocarrier suspension to a centrifuge tube and centrifuging at 75,600× g and 4 ° C. for 50 minutes, removing the supernatant, and re-suspending the pellet in DPBS containing 0.25% w/v PVA. The wash procedure is repeated and then the pellet re-suspended in DPBS containing 0.25% w/v PVA to achieve a nanocarrier suspension having a nominal concentration of 10 mg/mL on a polymer basis. The nanocarrier suspension is then filtered using a 0.22 μm PES membrane syringe filter from MilliporeSigma (EMD Millipore, 290 Concord Rd. Billerica MA, product code SLGP033RB). The filtered nanocarrier suspension is then stored at −20° C.

### Immunosuppressants

Any immunosuppressant as provided herein can be used in the methods or compositions provided and can be, in some embodiments, attached to, or comprised in, synthetic nanocarriers. Immunosuppressants include, but are not limited to, statins; mTOR inhibitors, such as rapamycin or a rapamycin analog; TGF-Iβ signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of statins include atorvastatin (LIPITOR®, TORVAST®), cerivastatin, fluvastatin (LESCOL®, LESCOL® XL), lovastatin (MEVACOR®, ALTOCO ®, ALTOPREV®), mevastatin (COMPACTIN®, pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®).

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, TX, USA).

Examples of TGF-β signaling agents include TGF-β ligands (e.g., activin A, GDF1, GDF11, bone morphogenic proteins, nodal, TGF-βs) and their receptors (e.g., ACVR1B, ACVR1C, ACVR2A, ACVR2B, BMPR2, BMPR1A, BMPR1B, TGFβRI, TGFIβRII), R-SMADS/co-SMADS (e.g., SMAD1, SMAD2, SMAD3, SMAD4, SMAD5, SMAD8), and ligand inhibitors (e.g, follistatin, noggin, chordin, DAN, lefty, LTBP1, THBS1, Decorin).

Examples of inhibitors of mitochondrial function include atractyloside (dipotassium salt), bongkrekic acid (triammonium salt), carbonyl cyanide m-chlorophenylhydrazone, carboxyatractyloside (e.g., from Atractylis gummifera), CGP-37157, (−)-Deguelin (e.g., from Mundulea sericea), F16, hexokinase II VDAC binding domain peptide, oligomycin, rotenone, Ru360, SFK1, and valinomycin (e.g., from Streptomyces fulvissimus) (EMD4Biosciences, USA).

Examples of P38 inhibitors include SB-203580 (4-(4-Fluorophenyl)-2-(4-methylsulfinylphenyl)-5-(4-pyridyl)1H-imidazole), SB-239063 (trans-1-(4hydroxycyclohexyl)-4-(fluorophenyl)-5-(2-methoxy-pyrimidin-4-yl) imidazole), SB-220025 (5-(2amino-4-pyrimidinyl)-4-(4-fluorophenyl)-1-(4-piperidinyl)imidazole)), and ARRY-797.

Examples of NF (e.g., NK-κβ) inhibitors include IFRD1, 2-(1,8-naphthyridin-2-yl)-Phenol, 5-aminosalicylic acid, BAY 11-7082, BAY 11-7085, CAPE (Caffeic Acid Phenethylester), diethylmaleate, IKK-2 Inhibitor IV, IMD 0354, lactacystin, MG-132 [Z-Leu-Leu-Leu-CH0], NFκB Activation Inhibitor III, NF-κB Activation Inhibitor II, JSH-23, parthenolide, Phenylarsine Oxide (PAO), PPM-18, pyrrolidinedithiocarbamic acid ammonium salt, QNZ, RO 106-9920, rocaglamide, rocaglamide AL, rocaglamide C, rocaglamide I, rocaglamide J, rocaglaol, (R)-MG-132, sodium salicylate, triptolide (PG490), and wedelolactone.

Examples of adenosine receptor agonists include CGS-21680 and ATL-146e.

Examples of prostaglandin E2 agonists include E-Prostanoid 2 and E-Prostanoid 4.

Examples of phosphodiesterase inhibitors (non-selective and selective inhibitors) include caffeine, aminophylline, IBMX (3-isobutyl-1-methylxanthine), paraxanthine, pentoxifylline, theobromine, theophylline, methylated xanthines, vinpocetine, EHNA (erythro-9-(2-hydroxy-3-nonyl)adenine), anagrelide, enoximone (PERFAN™), milrinone, levosimendon, mesembrine, ibudilast, piclamilast, luteolin, drotaverine, roflumilast (DAXAS™, DALIRESP™), sildenafil (REVATION®, VIAGRA®), tadalafil (ADCIRCA®, CIALIS®), vardenafil (LEVITRA®, STAXYN®), udenafil, avanafil, icariin, 4-methylpiperazine, and pyrazolo pyrimidin-7-1.

Examples of proteasome inhibitors include bortezomib, disulfiram, epigallocatechin-3-gallate, and salinosporamide A.

Examples of kinase inhibitors include bevacizumab, BIBW 2992, cetuximab (ERBITUX®), imatinib (GLEEVEC®), trastuzumab (HERCEPTIN®), gefitinib (IRESSA®), ranibizumab (LUCENTIS®), pegaptanib, sorafenib, dasatinib, sunitinib, erlotinib, nilotinib, lapatinib, panitumumab, vandetanib, E7080, pazopanib, and mubritinib.

Examples of glucocorticoids include hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (DOCA), and aldosterone.

Examples of retinoids include retinol, retinal, tretinoin (retinoic acid, RETIN-A®), isotretinoin (ACCUTANE®, AMNESTEEM®, CLARAVIS®, SOTRER®), alitretinoin (PANRETIN®), etretinate (TEGISON™) and its metabolite acitretin (SORIATANE®), tazarotene (TAZORAC®, AVAGE®, ZORAC®), bexarotene (TARGRETIN®), and adapalene (DIFFERIN®).

Examples of cytokine inhibitors include IL lra, IL1 receptor antagonist, IGFBP, TNF-BF, uromodulin, Alpha-2-Macroglobulin, Cyclosporin A, Pentamidine, and Pentoxifylline (PENTOPAK®, PENTOXIL®, TRENTAL®).

Examples of peroxisome proliferator-activated receptor antagonists include GW9662, PPARy antagonist III, G335, and T0070907 (EMD4Biosciences, USA).

Examples of peroxisome proliferator-activated receptor agonists include pioglitazone, ciglitazone, clofibrate, GW1929, GW7647, L-165,041, LY 171883, PPARγ activator, Fmoc-Leu, troglitazone, and WY-14643 (EMD4Biosciences, USA).

Examples of histone deacetylase inhibitors include hydroxamic acids (or hydroxamates) such as trichostatin A, cyclic tetrapeptides (such as trapoxin B) and depsipeptides, benzamides, electrophilic ketones, aliphatic acid compounds such as phenylbutyrate and valproic acid, hydroxamic acids such as vorinostat (SAHA), belinostat (PXD101), LAQ824, and panobinostat (LBH589), benzamides such as entinostat (MS-275), CI994, and mocetinostat (MGCD0103), nicotinamide, derivatives of NAD, dihydrocoumarin, naphthopyranone, and 2-hydroxynaphaldehydes.

Examples of calcineurin inhibitors include cyclosporine, pimecrolimus, voclosporin, and tacrolimus.

Examples of phosphatase inhibitors include BN82002 hydrochloride, CP-91149, calyculin A, cantharidic acid, cantharidin, cypermethrin, ethyl-3,4-dephostatin, fostriecin sodium salt, MAZ51, methyl-3,4-dephostatin, NSC 95397, norcantharidin, okadaic acid ammonium salt from prorocentrum concavum, okadaic acid, okadaic acid potassium salt, okadaic acid sodium salt, phenylarsine oxide, various phosphatase inhibitor cocktails, protein phosphatase 1C, protein phosphatase 2A inhibitor protein, protein phosphatase 2A1, protein phosphatase 2A2, and sodium orthovanadate.

Preferably, in some embodiments of any one of the methods or compositions or kits provided herein, the immunosuppressant is rapamycin. In some of such embodiments, the rapamycin is preferably encapsulated in the synthetic nanocarriers. Rapamycin is the active ingredient of Rapamune, an immunosuppressant which has extensive prior use in humans and is currently FDA approved for prophylaxis of organ rejection in kidney transplant patients aged 13 or older.

When coupled to a synthetic nanocarrier, the amount of the immunosuppressant coupled to the synthetic nanocarrier based on the total dry recipe weight of materials in an entire synthetic nanocarrier (weight/weight), is as described elsewhere herein. Preferably, in some embodiments of any one of the methods or compositions or kits provided herein, the load of the immunosuppressant, such as rapamycin or rapalog, is between 7% and 12% or 8% and 12% by weight.

### Compositions and Kits

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in a sterile saline solution for injection together with a preservative.

It is to be understood that the compositions of the invention can be made in any suitable manner, and the invention is in no way limited to compositions that can be produced using the methods described herein. Selection of an appropriate method of manufacture may require attention to the properties of the particular elements being associated.

In some embodiments, compositions are manufactured under sterile conditions or are initially or terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection. In some embodiments, the compositions may be lyophilized and stored in suspension or as lyophilized powder depending on the formulation strategy for extended periods without losing activity. The compositions referred to herein may be manufactured and prepared for administration using conventional methods.

The compositions of the invention can be administered in effective amounts, such as the effective amounts described elsewhere herein. Doses of compositions as provided herein may contain varying amounts of Ig protease fusion protein and/or synthetic nanocarriers comprising an immunosuppressant and/or other therapeutic according to the invention. The amount of elements present in the compositions for dosing can be varied according to their nature, the therapeutic benefit to be accomplished, and other such parameters. In some embodiments of any one of the methods or compositions provided herein, the doses of the Ig protease fusion protein and/or synthetic nanocarriers comprising an immunosuppressant and/or other therapeutic is each any one of the doses provided herein.

Another aspect of the disclosure relates to kits. In some embodiments, the kit comprises any one or more of the compositions provided herein. In some embodiments of any one of the kits provided, the kit comprises any one or more of the compositions comprising an Ig protease fusion protein as provided herein. Preferably, the Ig protease fusion protein comprising composition(s) is/are in an effective amount. The Ig protease fusion protein-comprising composition(s) can be in one container or in more than one container in the kit. In some embodiments of any one of the kits provided, the kit further comprises any one or more of the synthetic nanocarrier compositions and/or other therapeutics provided herein. Preferably, in some embodiments, the synthetic nanocarrier composition(s) is/are in an amount to provide one or more of the immunosuppressant doses provided herein. The Ig protease fusion protein and/or synthetic nanocarriers and/or other therapeutic can be in one container or in more than one container in the kit. In some embodiments of any one of the kits provided, the container is a vial or an ampoule. In some embodiments of any one of the kits provided, the composition(s) are in lyophilized form each in a separate container or in the same container, such that they may be reconstituted at a subsequent time. In some embodiments of any one of the kits, the lyophilized composition further comprises a sugar, such as mannitol. In some embodiments of any one of the kits provided, the composition(s) are in the form of a frozen suspension each in a separate container or in the same container, such that they may be reconstituted at a subsequent time. In some embodiments of any one of the kits, the frozen suspension further comprises PBS. In some embodiments of any one of the kits, the kit further comprises PBS and/or 0.9% sodium chloride, USP. In some embodiments of any one of the kits provided, the kit further comprises instructions for reconstitution, mixing, administration, etc. In some embodiments of any one of the kits provided, the instructions include a description of any one of the methods described herein. Instructions can be in any suitable form, e.g., as a printed insert or a label. In some embodiments of any one of the kits provided herein, the kit further comprises one or more syringes or other device(s) that can deliver the composition(s) in vivo to a subject.

### EXAMPLES

### Example 1: Synthesis of Engineered IdeS-mouse Fc Fusion Proteins

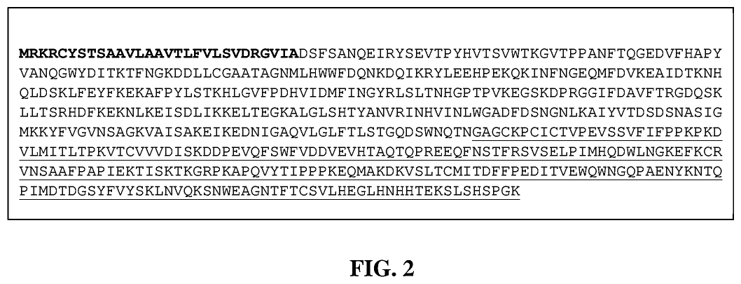

An IdeS-mouse Fc fusion protein was engineered to improve circulating half-life of an Ig protease for therapeutic potential (FIG. 2). The IdeS derived from Streptococcus pyogenes cleaves IgG from human, nonhuman primates and rabbits but not mouse IgG. The fusion protein was designed fusing the C-terminal end of IdeS (FIG. 2, normal text) with the N-terminal end of the mouse IgG1 Fc (FIG. 2, underlined text). A signal sequence was also included in the fusion protein (FIG. 2, bolded text).

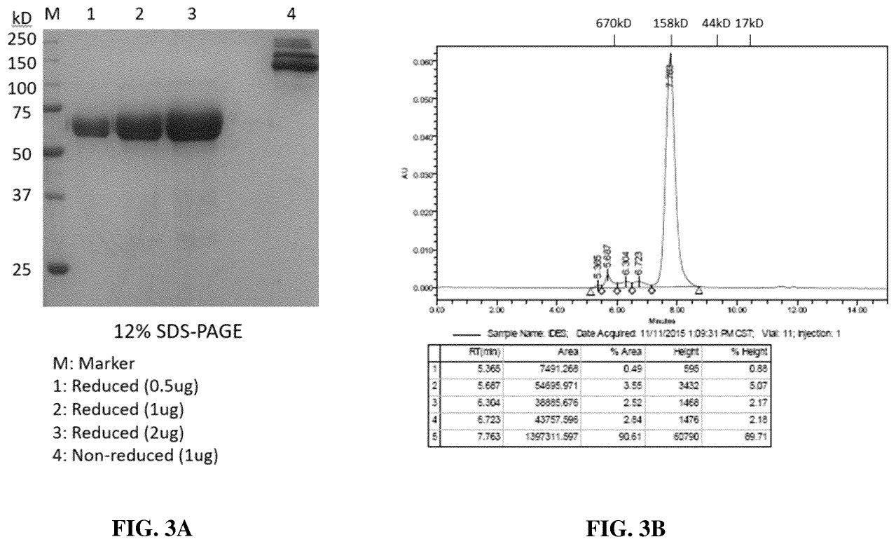

The IdeS-Fc fusion protein was cloned into a mammalian expression plasmid and transiently transfected into 293 cells. The cell supernatant was purified on a HiTrap MabSelect SuRe column, and the column was washed with 1.5M NaCl, 1.5M glycine, pH 8.5 and eluted with 50 mM Tris, 25 mM arginine, pH 10. The IdeS-Fc fusion protein eluted as a disulfide-linked homodimer of ˜120,000 daltons, which reduced to a single band of -60,000 daltons on a reducing SDS-PAGE gel (FIG. 3A). A native SEC-HPLC analysis of purified IdeS-Fc fusion protein was also performed. (FIG. 3B).

### Example 2: Activity of Engineered IdeS-mouse Fc Fusion Protein In Vivo

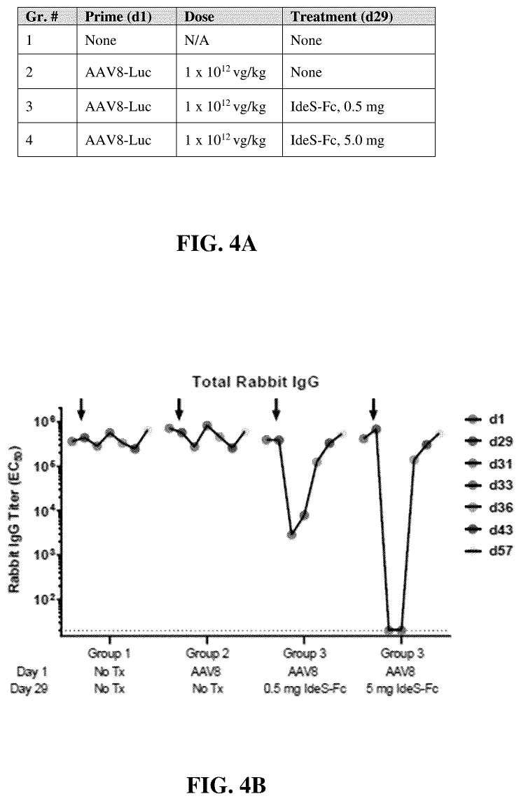

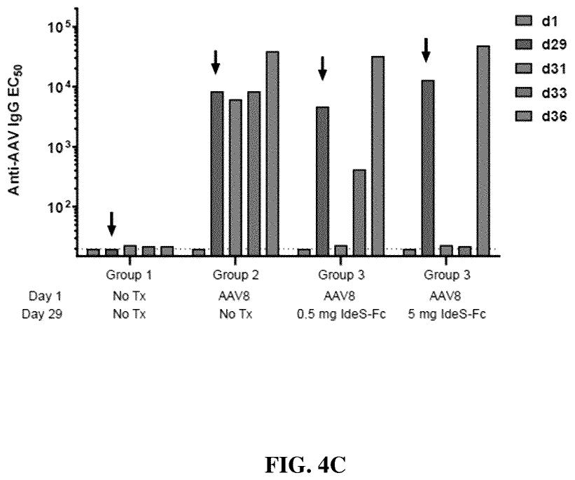

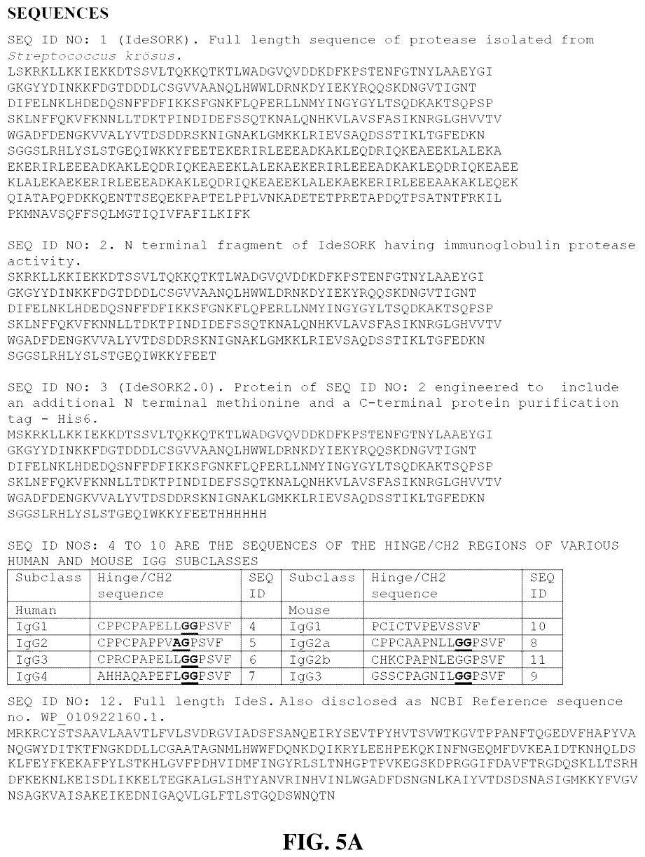

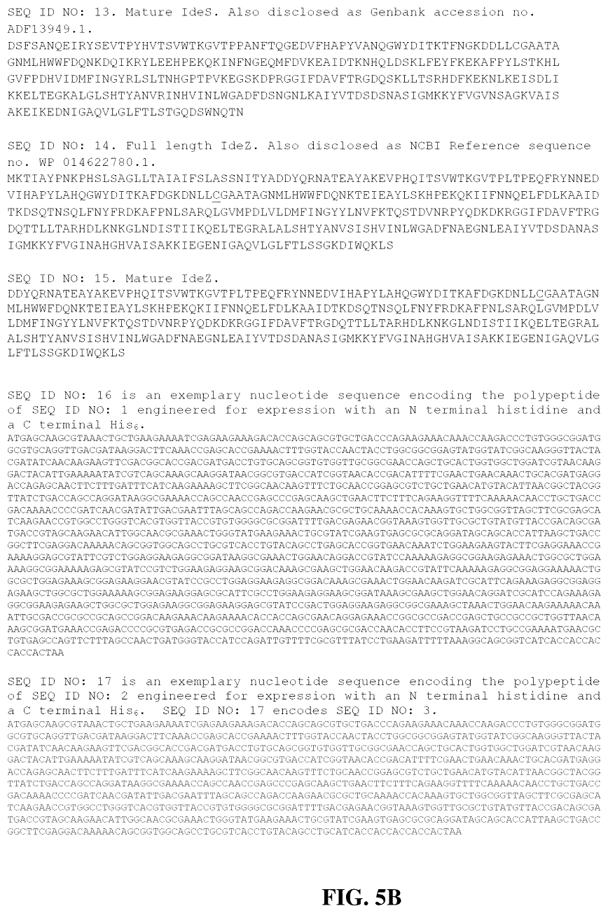

The IdeS-mouse Fc fusion protein was evaluated in vivo in rabbits. Rabbits were left untreated (Group 1) or immunized with 1×10¹² vector genomes/kg of AAV8 (Adeno-associated virus-8) on day 1 and then not treated (Group 2) or treated with 0.5 mg (Group 3) or 5 mg (Group 4) of IdeS-mouse Fc fusion protein on day 29. Control animals were left untreated (Group 1) (FIG. 4A). Total IgG was assessed after dosing of IdeS-Fc on day 29 (FIG. 4B). Total IgG dropped below detectable quantifiable levels by Day 31 and recovered by Day 36 in rabbits treated with 5 mg of Ides-Fc (FIG. 4B). Anti-AAV8 specific IgG showed a similar pattern (FIG. 4C).

### Example 3: Synthesis of Engineered IdeS-human Fc Fusion Protein (Prophetic)

For therapeutic use in humans, it is contemplated that it would be advantageous to use human Fc rather than mouse Fc. However, as IdeS cleaves human IgG, an IdeS-Fc fusion protein would have to be mutated to be resistant to autoproteolysis. IdeS cleaves human IgG near the border of the hinge region and the CH2 domain. The P1, P2, P1′, and P2′ residues of the IdeS cleavage site are L, G, G, and P, respectively, in human IgG1, IgG3 and IgG4, and V, A, G, and P in human IgG2 (Wenig K, et al. Structure of the streptococcal endopeptidase IdeS, a cysteine proteinase with strict specificity for IgG. Proc Natl Acad Sci U S A. 2004 Dec 14;101(50):17371-6. doi: 10.1073/pnas.0407965101). However, IdeS does not cleave short synthetic peptides that span the cleavage site, indicating a secondary docking on IgG that provides specificity (Vincents B, et al. Enzymatic characterization of the streptococcal endopeptidase, IdeS, reveals that it is a cysteine protease with strict specificity for IgG cleavage due to exosite binding. Biochemistry. 2004 Dec. 14;43(49):15540-9. doi: 10.1021/bi048284d). However, human CH2 fusion proteins are cleaved by IdeS, indicating there is a binding within the CH2 domain (Novarra S, et al. A hingeless Fc fusion system for site-specific cleavage by IdeS. MAbs. 2016 August-September;8(6):1118-25. doi: 10.1080/19420862.2016.1186321). Thus, mutations are contemplated by the present invention that block autoproteolysis of an IdeS-human Fc fusion protein, which could be made in the cleavage site, near the border of the hinge region and the CH2 domain, or within the putative docking site in the CH2.

Analysis of the crystal structure of IdeS overlaid on the homologous structure of papain indicates that the carbonyl oxygen of the IgG P1 residue (Gly-236) forms hydrogen bonds with the peptide bond amide of Cys-94 and the side-chain nitrogen of Lys-84 of IdeS (Wening PNAS 2004). This orientation directs the amide nitrogen of IdeS P1′ residue (Gly-237) close to ND1 of the imidazole of His-262. The putative S1′ subsite in IdeS is narrower than that of papain, and suggests that P1′ residues larger than glycine may provide steric hinderance (Wening et al PNAS 2004).

In some embodiments, an IdeS protease domain may be replaced with mutated, truncated or engineered versions that have one or more desired activities and/or functions. In some embodiments, an IdeS protease domain may be replaced by homologous protease domains from other strains of streptococcal bacterial, such as an IdeZ protease domain or mutated, truncated or engineered versions thereof. In some embodiments, an IdeS protease domain may be replaced by an IdeMC protease domain (U.S. Patent Application No. 20190262434 A2), an IgG protease domain produced by a canine-specific mycoplasma strain, or by mutated, truncated or engineered versions of an IdeMC protease domain.

In some embodiments, the Fc domain may be derived from human IgG1, IgG2, IgG3, or IgG4 or versions that have been mutated or engineered, for example to reduce or eliminate Fc effector functions or complement fixation or to enhance binding to FcRn. In some embodiments, the hinge region of the Fc domain may be mutated, truncated or deleted.

### Example 4: Synthesis of Engineered Ig Protease-albumin Fusion Protein (Prophetic)

In some embodiments, non-natural Ig proteases may be engineered to comprise albumin, a plasma protein that also has a long circulating half-life mediated through FcRn binding. In some embodiments, the albumin of a non-natural Ig protease may be a human albumin protein. In some embodiments, the non-natural Ig protease can comprise albumin and an Ig protease domain of IdeS from Streptococcus pyogenes.

In some embodiments, the IdeS protease domain may be replaced with mutated, truncated or engineered versions that have one or more desired activities and/or functions. In some embodiments, the IdeS protease domain may be replaced by homologous protease domains from other strains of streptococcal bacterial, such as of IdeZ or mutated, truncated or engineered versions thereof. In some embodiments, the IdeS protease domain may be replaced by a protease domain of IdeMC (e.g., U.S. Patent Application No. 20190262434 A2), an IgG protease domain produced by a canine-specific mycoplasma strain, or by mutated, truncated or engineered versions of an IdeMC protease domain.

### Example 5: Administration of Engineered Ig Protease Fusion Protein (Prophetic)

Ig protease fusion proteins can be administered to a subject according to any one of the methods known to a person of ordinary skill in the art. In some embodiments, routes of administration include oral or intravenous administration. In some embodiments, routes of administration may be localized. In other embodiments, routes of administration may be systemic. It is contemplated that subjects can have any one of the diseases, disorders or conditions provided herein.

### Example 6: Xork-Fc fusion homodimer can be expressed in CHO and E. Coli

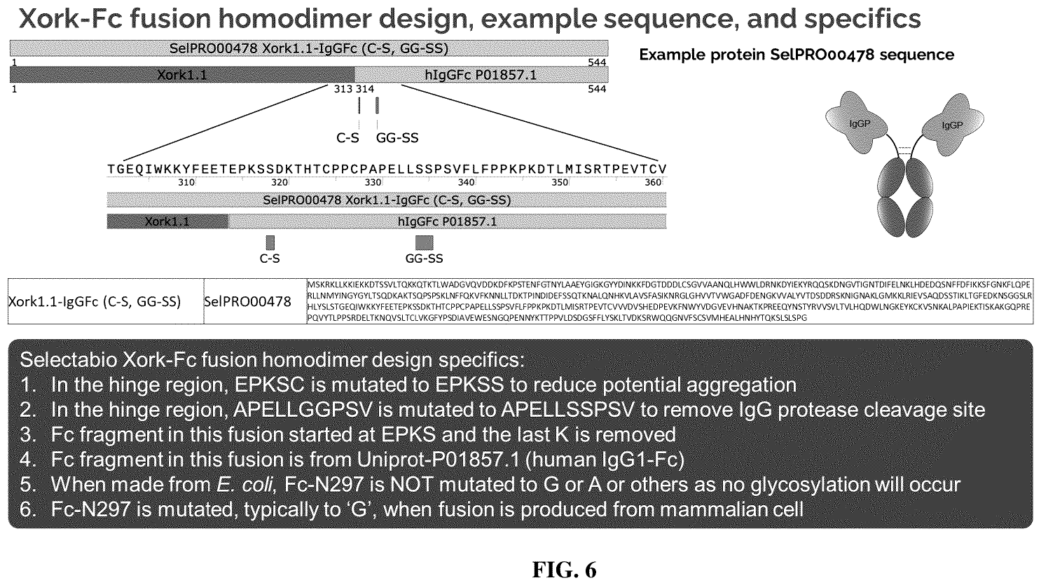

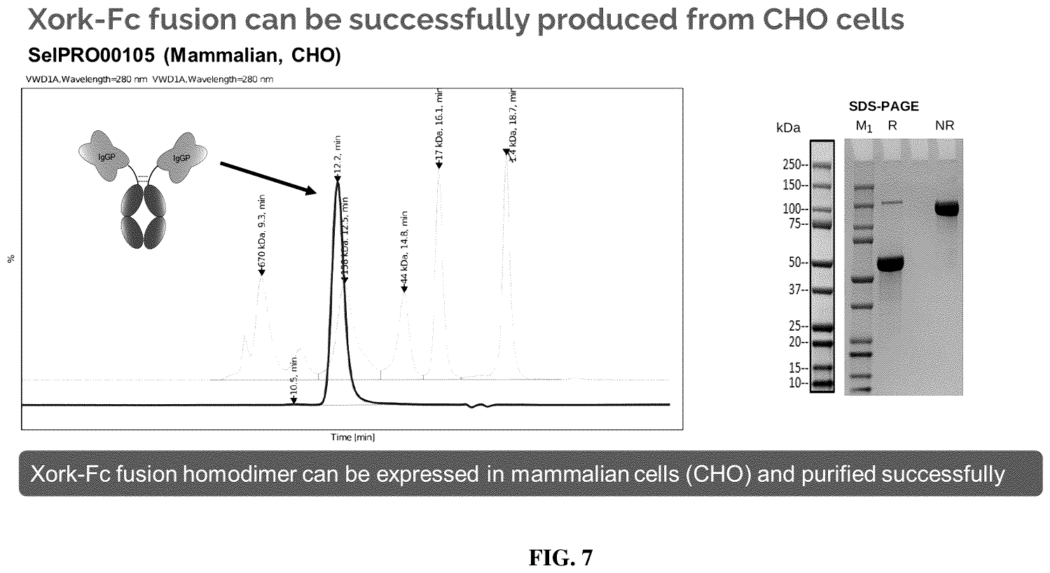

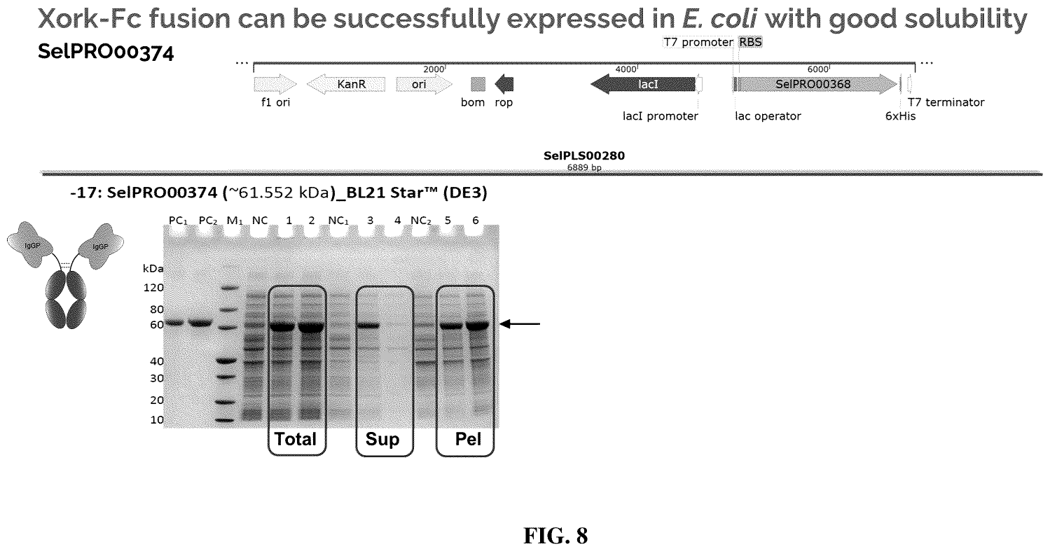

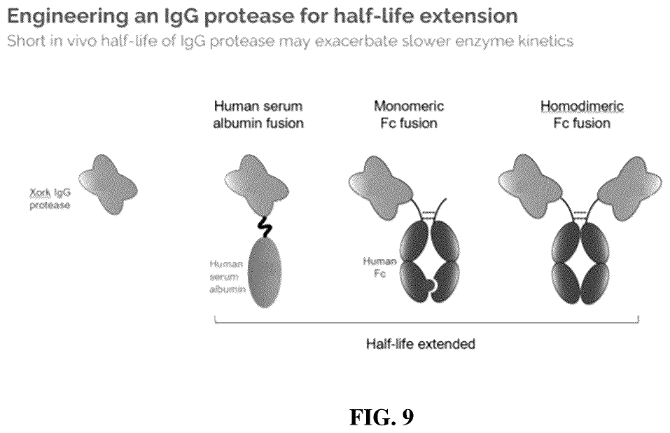

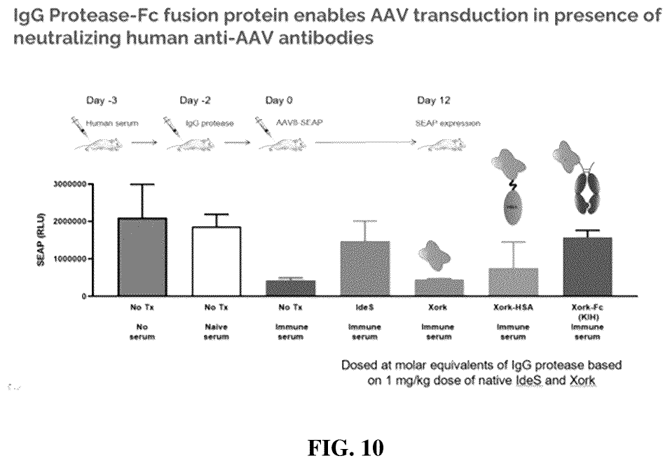

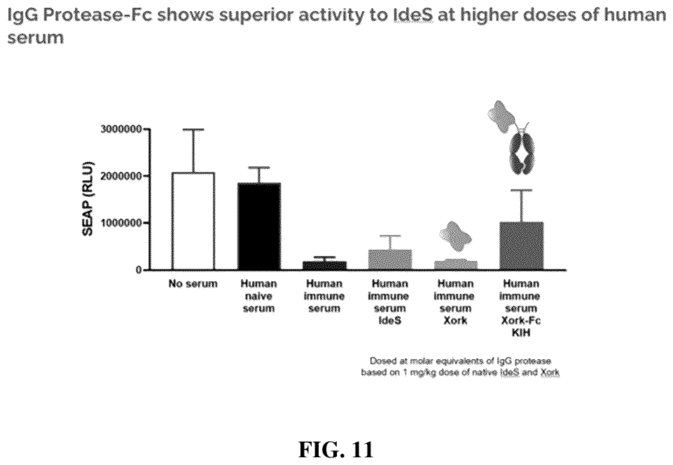

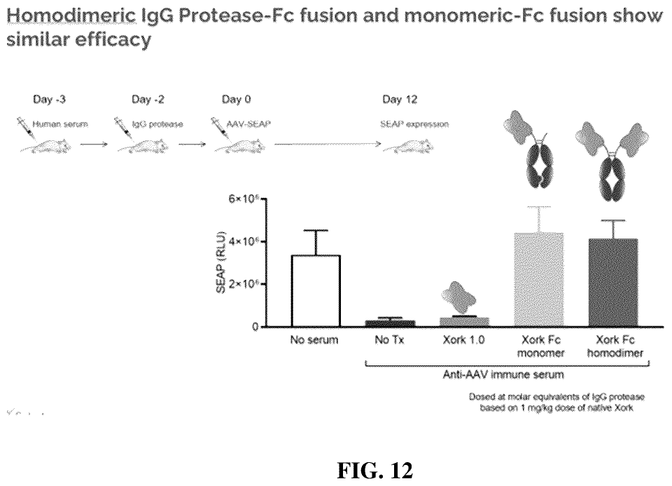

An Ig protease (Xork)-Fc fusion was designed as described in FIG. 6. It has been found, as an example, that an Ig protease (Xork)-Fc fusion can be expressed in CHO cells successfully (FIG. 7). It was further found that an exemplary Ig protease (Xork)-Fc fusion could be prepared from E. coli cells (FIG. 8).

Example 7: Xork-Fc, an Engineered IgG Protease, Shows Low Cross Reactivity to Pre-existing Antibodies in Human Serum and Enables Efficient AAV Transduction in an In Vivo Model of Passive Transfer of Neutralizing Human Serum

Pre-existing neutralizing antibodies against AAV are highly prevalent and are a major exclusion factor for enrollment into many gene therapy trials. Strategies are needed to expand access of critical gene therapies to patients with pre-existing anti-AAV antibodies. Recently, bacterial-derived proteases specific for human IgG have been proposed as a method to transiently clear IgG from circulation and open a window within which AAV can be administered. A novel IgG-specific protease, IdeSork (Xork), has been developed. In contrast to IdeS, an IgG protease derived from the common human pathogen Streptococcus pyogenes, Xork is derived from a Streptoccocus species that is not known to infect humans. Consequently, levels of pre-existing antibodies against Xork are low or absent in normal human serum compared to the moderate-high levels of pre-existing antibodies that are prevalent against IdeS. Xork cleaves human IgG in vitro with the same specificity and mechanism as IdeS. The in vivo activity of Xork was optimized by creation of an Fc fusion protein to extend its half-life. Inhibition of AAV transduction in vivo by passive transfer of human serum with pre-existing anti-AAV antibodies was efficiently prevented by treatment with Xork-Fc. Combining Xork-Fc with ImmTOR tolerogenic nanoparticles can prevent de novo formation of anti-Xork antibodies and enable re-dosing of Xork.

### Example 8: Administration of Engineered Ig Protease-Fc Fusion Protein in Combination with Synthetic Nanocarriers Comprising Rapamycin (e.g, ImmTOR) (Prophetic)

Ig protease domain-Fc fusion proteins can be administered in combination with synthetic nanocarriers comprising rapamycin to a subject according to any one of the methods provided herein or known to a person of ordinary skill in the art. In some embodiments, routes of administration include oral or intravenous administration. In some embodiments, routes of administration may be localized. In other embodiments, routes of administration may be systemic. It is contemplated that subjects can have any one of the diseases, disorders or conditions provided herein.

### Example 9: Synthesis of Synthetic Nanocarriers Comprising an Immunosuppressant (Prophetic)

Synthetic nanocarriers comprising an immunosuppressant, such as rapamycin, can be produced using any method known to those of ordinary skill in the art. Preferably, in some embodiments of any one of the methods or compositions provided herein the synthetic nanocarriers comprising an immunosuppressant are produced by any one of the methods of US Publication No. US 2016/0128986 A1 and US Publication No. US 2016/0128987 A1, the described methods of such production and the resulting synthetic nanocarriers being incorporated herein by reference in their entirety. In any one of the methods or compositions provided herein, the synthetic nanocarriers comprising an immunosuppressant are such incorporated synthetic nanocarriers.

### Example 10: Xork IgG Protease Candidate Molecules In Vivo Activity Testing (Mitigation of AAV Neutralization by Human Immune Serum in Passively-Immunized Mice)

### Materials and Methods

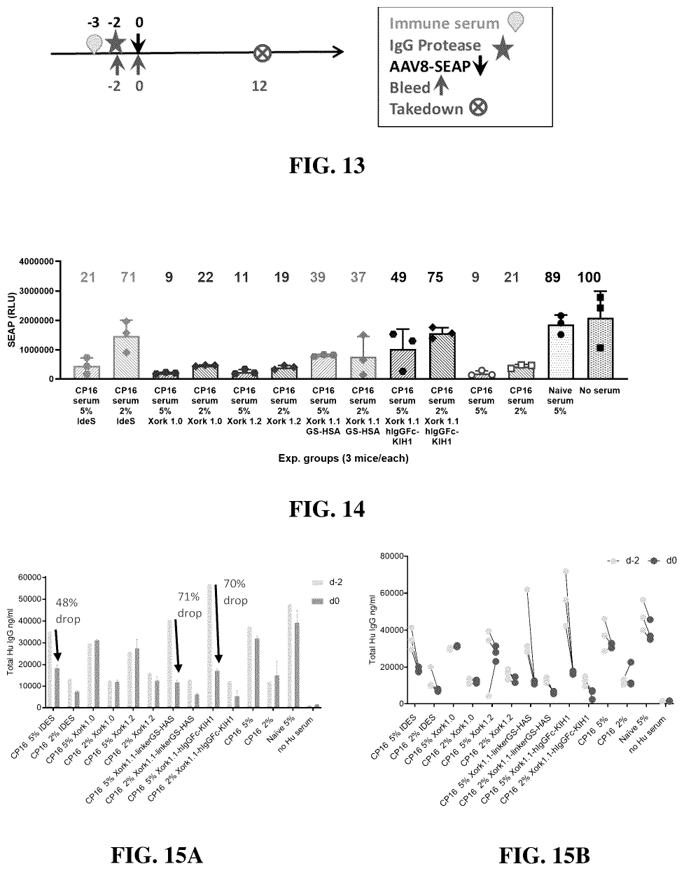

Experimental outline. The experiments utilized immunologically naive female C57BL/6 female mice (17g, 3-4/group). Pre-tested AAV8 positive human serum (pooled samples CP16 or P1) or control normal serum was diluted 5-100-fold (so the final injectable solution had 1-20% of original serum) and diluted serum was then heat-inactivated at 56° C. for 30 minutes prior to injection of 100 μL per mouse (i.v.; retro-orbital). One control group was left non-injected or was injected with PBS only. Twenty-four hours (1 day) after immune serum injection, mice were either mock-treated or injected (i.v.; retro-orbital, contralateral site with respect to previous injection) with equimolar amounts of various Xork IgG protease molecules with a typical dose being 2.095E⁻⁰⁸ M/kg. Two days after IgG protease treatment all mice in the study were injected with AAV8-SEAP at 5E¹¹ vg/kg (i.v., r.o.). Mice were bled 12 days after AAV inoculation, and SEAP activity in serum was determined. Timeline of a typical study is shown in FIG. 13.

Assessment of results. The efficacy of protease molecules to cleave human IgG was measured in vivo in mice passively immunized against AAV8. In this setting mice were administered human serum containing IgG against AAV8 and then injected with AAV8 carrying a reporter gene, secreted alkaline phosphatase (SEAP). If no protease treatment was administered, the level of AAV vector transduction as measured by SEAP expression was significantly (up to 20-fold) lower than in naive, non-immunized mice. Thus, the activity of protease (administered 1 day after serum transfer and two days prior to AAV inoculation) was measured by its ability to elevate AAV-encoded transgene activity vs. that in passively immunized untreated animals. The results were expressed relative to SEAP activity in mice not injected with human immune serum (naïve animals) as follows. Average SEAP activity in the group mock-immunized with PBS or immunized with normal (non-immune) human serum and then left untreated with IgG protease was regarded as 100% with activity in all other experimental groups expressed as % with respect to this group. One group was always injected with immune serum and then not treated with protease with resulting SEAP activity (usually within 5-10% from non-immunized control) indicating the maximum level of AAV transduction inhibition seen in this study (i.e., usually in 90-95% range). SEAP activity in the experimental group being statistically indistinguishable from that of mock-immunized mice was regarded as an indicator of full cleavage of human anti-AAV IgG by molecule tested, SEAP activity lower than in mock-injected control but higher than in protease-untreated immunized controls was regarded as indicator of partial IgG cleavage by molecule tested and activity insignificantly different from protease-untreated immunized control was regarded as indicator of no or insufficient human IgG cleavage.

SEAP activity measurement. SEAP expression was determined using the Phospha-Light SEAP Reporter Gene Assay System (Invitrogen, Carlsbad, CA). Samples were diluted 1:10, heat-inactivated (65° C., 30 min) and cooled over ice. Once the samples reached room temperature, they were added to opaque white assay plates, followed by the assay buffer (5 min) and substrate (20 min) per the manufacturer's recommendations. Luminescence was read at 477 nm on SpectraMax L (Molecular Devices, San Jose, CA, USA) and reported in relative luminescence units (RLUs), which are proportional to the concentration of SEAP in the serum.

AAV IgG ELISA. In certain studies, levels of total IgG or IgG against AAV8 at different time-points were assayed by ELISA and expressed as top OD or by AAV8 neutralization assay (expressed as EC50). In a particular iteration, groups of mice were bled either immediately prior to IgG protease injection (to determine total IgG or AAV antibody levels at treatment initiation) or two days later, immediately prior to AAV inoculation (to determine total IgG or AAV antibody levels after IgG protease treatment). AAV antibody level was also determined in positive human serum samples then pooled and used for passive immunization. For ELISA analysis, the 96-well plates were coated o/n with AAV8 or anti-human IgG capture antibody, washed, and blocked on the following day, followed by sample incubation (1:40 diluted serum). Plates were then washed, and the presence of IgG was detected using anti-human IgG-specific HRP detector (1:2000; Southern Biotech, Birmingham, AL, USA), with the presence of HRP being visualized using trimethylboron substrate and measured using absorbance at 450 nm with a reference wavelength of 570 nm. The optical density (OD) observed is proportional to the amount of anti-AAV8 human IgG antibody in a sample and was reported. Serum EC50 was determined as follows. The positive control anti-AAV8-IgG antibody (Fitzgerald Industries International, Acton, MA) and samples were diluted 1:40 followed by a 1:3 serial dilution. Plates were then processed as described above, and the EC50 was calculated using the four-parameter logistic curve fit function in the Softmax Pro software program (Molecular Devices, San Jose, CA). The positive control anti-AAV8—IgG antibody was used as the standard curve to determine the EC₅₀ for each sample.

PK Assay. ELISA plates were coated overnight at 4° C. with a custom polyclonal goat anti-Xork antibody at 1 μg/mL. The polyclonal anti-Xork antibody is able to capture Xork containing molecules (Xork 1.1 homodimer, Xork1.1 Fc H435R, Xork1.1 E. coli, Xork H435R E. coli and Xork1.1 IgG3Fc). After washing with PB ST, wells were blocked with 1% casein for 1 to 2 hours at room temperature. Plates were washed with PBST before the addition of samples. Samples were incubated for 2 hours at room temperature allowing for the capture of Xork-containing molecules. Plates were then washed with PBST before the addition of mouse anti-human IgG Fc-HRP. The secondary antibody detected the presence of human Fc captured to the wells. A final wash with PBST was then performed. Intact molecules (containing both Xork and a human Fc) were visualized using 3,3′,5,5′-Tetramethylbenzidine (TMB) which was oxidized by HRP conjugated to the mouse anti-human IgG Fc secondary antibody. The reaction was then stopped using sulfuric acid. Plates were then read at 450 and 570 nm using a spectrophotometer. The optical density (OD) of a sample (570 nm OD subtracted from 450 nm OD) was used to interpolate the concentration of intact Xork molecule, using the appropriate standard curve (Xork 1.1 homodimer, Xork1.1 Fc H435R, Xork1.1 E. coli, Xork H435R E. coli and XORK1.1 IgG3Fc), accounting for any sample dilution performed.

### Results

Experiment 1. The activity of Xork candidate molecules is elevated by their fusion to human IgG Fc domain with resulting levels of human IgG cleavage indistinguishable from that of IdeS protease. Twelve groups of three mice each were inoculated with 2% or 5% CP16 immune serum pool as described above, two more groups were either injected with naïve (non-immune) serum or mock-injected. Five pairs of experimental groups (each receiving 2% or 5% immune serum) were treated with IgG proteases as described above, inoculated with AAV8-SEAP two days later and SEAP activity in serum measured 12 days after AAV inoculation. One pair of experimental groups was not treated with protease prior to AAV injection in order to assess the levels of inhibition of AAV transduction. Molecules tested were clinically-approved IgG protease from Streptococcus pyogenes (IdeS) and four Xork candidate molecules: Xork1.0, Xork1.2 and two fusion molecules carrying Xork1.0 attached either to human serum albumin (HSA) or human IgG Fc domain (knob-in-hole structure, KIH), the latter two constructs designated as Xork1.1-HSA and Xork1.1-hIgGFc-KIH. Passive immunization with immune serum led to significant decrease of transduction efficiency as documented by SEAP activity in these groups being at 21% and 9% of untreated controls (after treatment with 2% and 5% serum, correspondingly, FIG. 14). Treatment with Xork1.0 and Xork 1.2 resulted in no elevation of SEAP activity compared to untreated control, and treatment with Xork1.1-HSA was partially efficient (FIG. 14). Conversely, treatment with Xork1.1-hIgGFc-KIH resulted in significant elevation of transduction activity as measured by SEAP expression with levels attained being indistinguishable from those seen in mice treated with IdeS protease. Furthermore, the levels of human IgG measured prior to and two days after administration of protease molecules provided a full complementation of SEAP activity data indicating the most profound decrease of total human IgG in groups treated with Xork1.1 and IdeS with virtually no IgG decrease seen in groups treated with Xork 1.0 or Xork1.2 (FIG. 15).

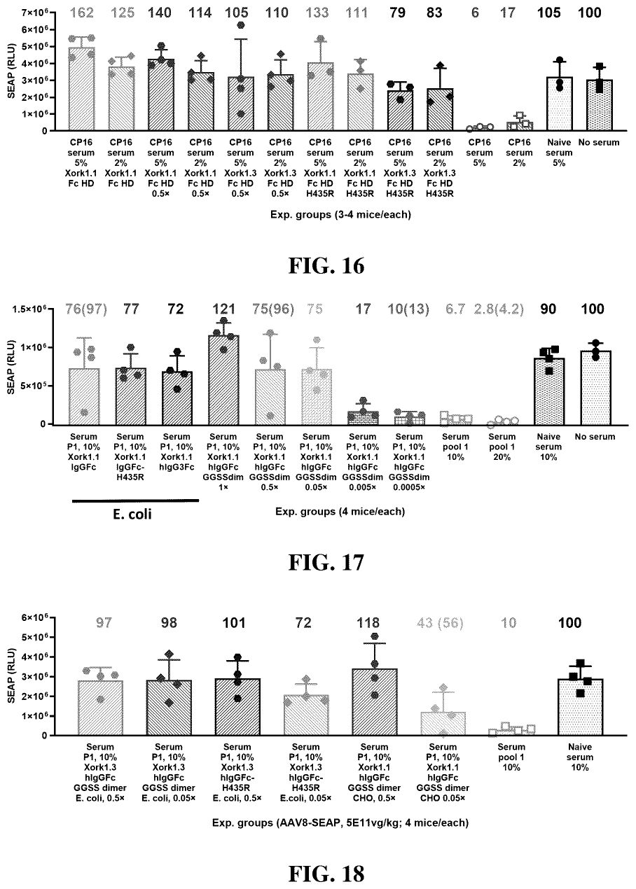

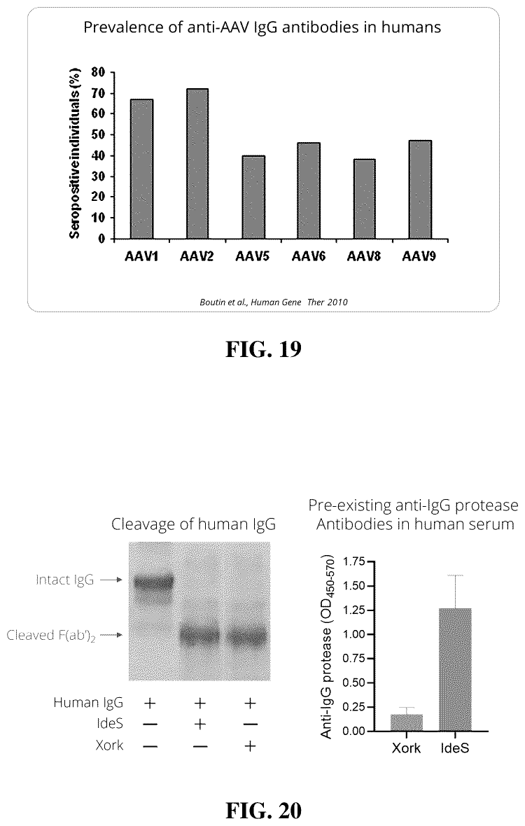

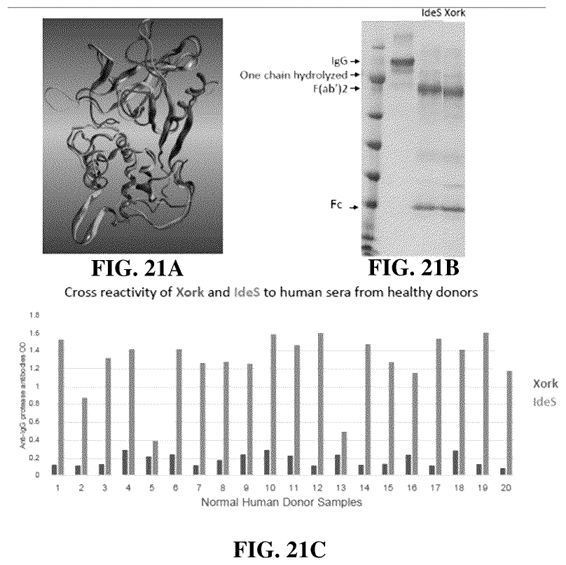

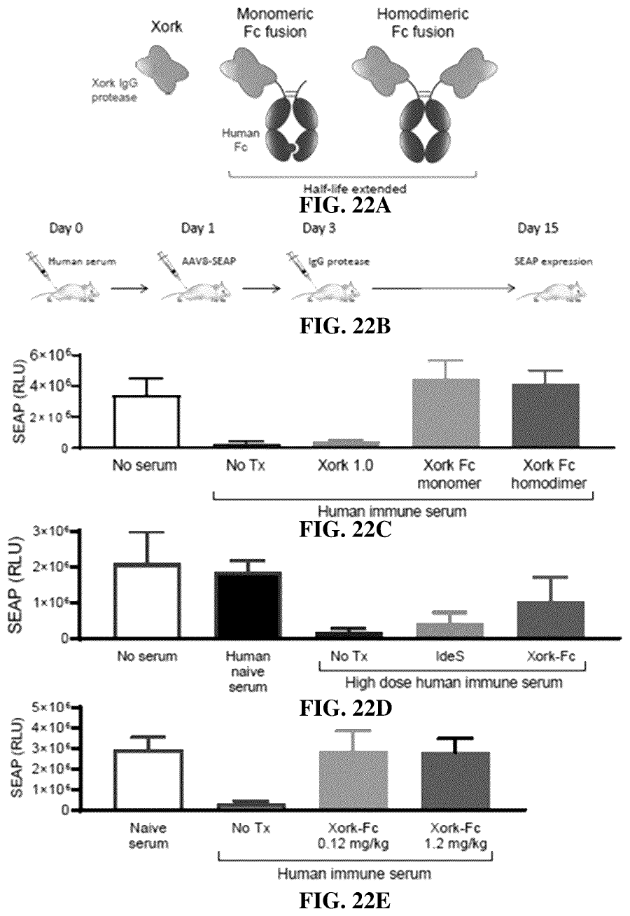

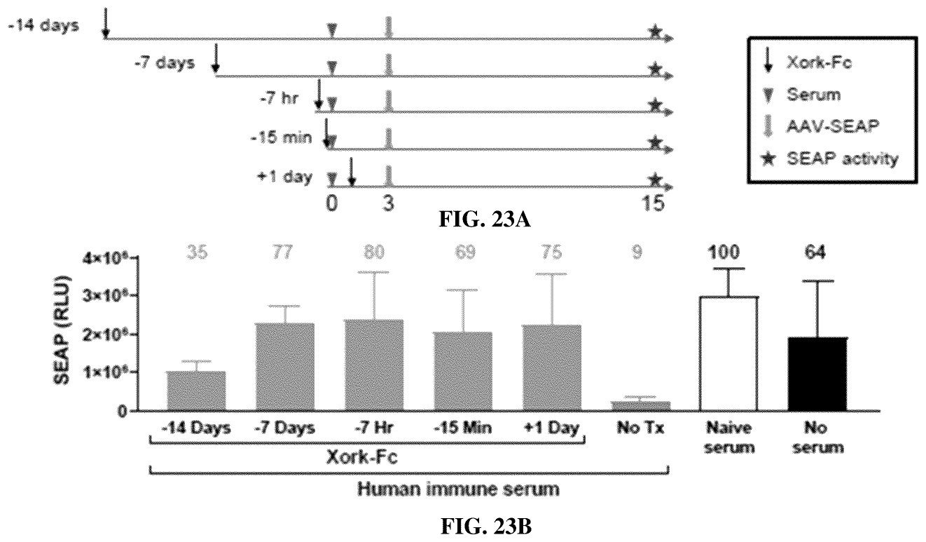

Experiment 2. Xork1.1 and Xork1.3 candidate molecules enable full AAV transduction activity in passively immunized mice at the standard and decreased dose of protease. Twelve groups of three or four mice each were inoculated with 2% or 5% CP16 immune serum pool as described above, two more groups were either injected with naïve (non-immune) serum or mock-injected. Five pairs of experimental groups (each receiving 2% or 5% immune serum) were treated with Xork1.1 or Xork1.3 IgG proteases (fusion constructs, consisting of Xork catalytic domain and human IgG Fc domain). Then they were inoculated with AAV8-SEAP two days later and SEAP activity in serum measured 12 days after AAV inoculation. One pair of experimental groups was not treated with protease prior to AAV injection to assess the levels of inhibition of AAV transduction. Furthermore, while groups 1-2, 7-8 and 9-10 were treated with the standard 2.095E⁻⁰⁸ M/kg dose of Xork1.1-Fc (homodimer or HD), Xork1.1-Fc-HD-H435R and Xork1.3-Fc-HD-H435R, respectively (the latter two bearing a single amino acid mutation in the Fc domain as indicated), groups 3-4 and 5-6 were treated with 2 times lower dose (1.048E⁻⁰⁸ M/kg) of Xork1.1-Fc-HD and Xork1.3-Fc-HD, respectively. In nearly all groups of mice treated with Xork1.1-Fc or Xork-1.3-Fc the level of SEAP expression was at least equal to or higher than that in control groups receiving no passive immunization (FIG. 16). This indicated high IgG cleaving efficacy of these molecules since protease-untreated group receiving 5% of AAV immune serum exhibited 94% decrease of SEAP activity (FIG. 16). The only test molecule which did not provide for a full AAV transduction efficacy was Xork1.3-Fc-HD-H435R, although SEAP levels in mice treated with construct were only ˜20% lower than that in non-immunized controls (FIG. 16).

Experiment 3. Activity of Xork1.1-hIgGFc-GGSS in vivo is not influenced by means of its manufacturing, it is exhibited over the wide dose range and may be superior to that of Xork1.1-IgGFc-H435R and Xork1.1-hIgG3-Fc. Nine groups of four mice each were inoculated with 10% P1 human immune serum pool as described earlier for CP16 pool, one group received a higher, 20% serum dose and two more groups were either injected with naïve (non-immune) serum or mock-injected. Eight experimental groups were treated with three different Xork1.1 molecules produced in E. coli (gr. 1-3) or with serially-diluted Xork1.1-hIgGFc-GGSS dimer produced in eukaryotic CHO cells (gr. 4-8; no dilution or standard 2.095E⁻⁰⁸ M/kg dose and its 2-, 20-, 200- and 2,000-fold dilution, respectively). Then they were inoculated with AAV8-SEAP two days later and SEAP activity in serum measured 12 days after AAV inoculation. Two groups (one inoculated with 10% immune serum and the other inoculated with 20% immune serum) were not treated with protease prior to AAV injection in order to assess the levels of inhibition of AAV transduction.

Levels of AAV transduction enabled by Xork1.1-hIgGFc-GGSS dimer produced in CHO cells appear slightly superior, but the activity of this molecule when diluted 2-fold is very similar to Xork1.1-IgGFc fusion, its E. coli-produced counterpart, indicating these two molecules being of similar potency (FIG. 17). It also appears possible that the activity of these molecules is significantly better than that of Xork1.1-IgGFc-H435R and Xork1.1-IgG3Fc since 20-fold lower dose of Xork1.1-hIgGFc in gr. 6 has resulted in similar SEAP activity to that in gr. 2 and gr. 3. Additionally, the decrease in activity by 2- and 20-fold diluted Xork1.1-hIgGFc-GGSS is not dramatic, dilutions of >20-fold (i.e., decreasing the dose lower than 1.048E⁻⁰⁹ M/kg ) are necessary to observe a noticeable transduction drop. Even if Xork1.1-hIgGFc-GGSS is diluted 200-2,000-fold (gr. 7, gr. 8) or to 1.048E⁻¹⁰ 1.048E¹¹ M/kg, the resulting SEAP activity is higher than that in protease-untreated control (gr. 9).

Experiment 4. Assessment of in vivo activity of Xork1.3-hIgGFc-GGSS homodimer produced in E. coli and CHO cells vs. E. coli-produced Xork1.3-hIgGFc-GGSS-H435R. Seven groups of four mice each were inoculated with 10% P1 immune serum pool and one group received naïve (non-immune) serum. Six experimental groups were treated either with Xork1.3-hIgGFc-GGSS-homodimer molecules produced in E. coli (gr. 1-2) or CHO cells (gr. 5-6) or treated with Xork1.3-hIgGFc-GGSS-H435R produced in E. coli (gr. 3-4). Each molecule was used in two doses, 1.048E⁻⁰⁸ M/kg and 1.048E⁻⁰⁹ M/kg (0.5× and 0.05× of the standard 2.095E⁻⁰⁸ M/kg dose, respectively). Then mice were inoculated with AAV8-SEAP two days later and SEAP activity in serum measured 12 days after AAV inoculation. One group inoculated with 10% immune serum was not treated with protease prior to AAV injection to assess the levels of inhibition of AAV transduction.

Similarly to its Xork1.1 counterpart, Xork1.3-hIgGFc-GGSS homodimer enables ˜100% AAV transduction in passively immunized mice with SEAP activity level in treated animals being indistinguishable from that injected with naïve, non-immune serum (FIG. 18). This activity is seen over the broad concentration range, even at 20-fold dilution vs. standard 2.095E⁻⁰⁸ M/kg dose. Moreover, at this low 1.048E⁻⁰⁸ M/kg dose Xork1.3-hIgGFc-GGSS-HD from E. coli enables the transduction levels, which are higher than those attained by other constructs tested in this study, namely Xork1.3-hIgGFc-H435R and from E. coli and CHO-made Xork1.3-hIgGFc-GGSS -HD. In any case, CHO-made Xork1.3-hIgGFc-GGSS -HD has shown no superiority in human IgG cleavage vs. its E. coli-produced counterpart in the in vivo passive immunization model.

### Example 11: Xork-Fc, an Engineered IgG protease, Shows Low Crossreactivity to Pre-existing Antibodies in Human Serum and Enables Efficient AAV Transduction in an In Vivo Model of Passive Transfer of Neutralizing Human Serum

Pre-existing neutralizing antibodies against AAV are highly prevalent and are a major exclusion factor for enrollment into many gene therapy trials. Individuals may not be able to be treated with viral vectors, such as AAV-mediated gene therapies, due to prior exposure neutralizing antibodies. New strategies would be helpful to expand access of critical gene therapies to patients with pre-existing anti-AAV antibodies.

Bacterial-derived proteases specific for human IgG can be a method to transiently clear IgG from circulation and open a window within which AAV can be administered. For example, a novel IgG-specific protease, IdeSork (Xork), has been developed. In contrast to IdeS, an IgG protease derived from the common human pathogen Streptococcus pyogenes, Xork is derived from a Streptoccocus species that is not known to infect humans. Consequently, levels of pre-existing antibodies against Xork are low or absent in normal human serum compared to the moderate-high levels of pre-existing antibodies that are prevalent against IdeS.

The protease cleaves human IgG specifically and efficiently and shows low cross reactivity to human sera. Xork has been demonstrated to cleave human IgG in vitro with the same specificity and mechanism as IdeS. The in vivo activity of Xork was optimized by creation of an Fc fusion protein to extend its half-life. Near-complete (up to 97%) inhibition of AAV transduction in vivo by passive transfer of human serum with pre-existing anti-AAV antibodies was efficiently prevented by treatment with Xork-Fc. Combining Xork-Fc with ImmTOR tolerogenic nanoparticles can prevent de novo formation of anti-Xork antibodies and enable re-dosing of Xork.

Thus, as provided herein the Ig protease fusion proteins (e.g., Xork-Fc)+synthetic nanocarriers comprising an immunosuppressant (e.g., ImmTOR) can address two major challenges in gene therapy: 1) increasing the number of patients eligible for gene therapy by mitigating pre-existing anti-AAV antibodies and 2) enabling re-dosing by mitigating the de novo formation of anti-AAV antibodies. Thus, provided herein are methods of administering both synthetic nanocarriers comprising an immunosuppressant and an Ig protease (e.g., IgG protease) fusion protein as provided herein to subjects that are also administered a viral vector therapy. Related compositions are also provided.

1. A composition comprising an Ig protease fusion protein, comprising:
(i) an Ig protease domain, and
(ii) an Fc domain, optionally, wherein the Ig protease fusion protein has an increased circulating half-life relative to a naturally occurring Ig protease.

2. A composition comprising an Ig protease fusion protein, comprising:
(i) an Ig protease domain, and
(ii) an albumin protein;
optionally, wherein the Ig protease fusion protein has an increased circulating half-life relative to a naturally occurring Ig protease.

3. The composition of claim 1, wherein the Ig protease fusion protein binds to a region of a target immunoglobulin, and wherein the Ig protease fusion protein cleaves the target immunoglobulin.

4. The composition of claim 3, wherein the Ig protease domain cleaves the target immunoglobulin in a hinge region of the target immunoglobulin.

5. The composition of claim 3, wherein the target immunoglobulin is IgG or IgA.

6. The composition of claim 1, wherein the Ig protease domain is from an Ig protease from a bacterial strain.

7. The composition of claim 6, wherein the bacterial strain is a Streptococcal bacterial strain.

8. The composition of claim 7, wherein the Streptococcal bacterial strain is Streptococcus pyogenes.

9. The composition of claim 8, wherein the Streptococcal bacterial strain is Streptococcus equii.

10. The composition of claim 6, wherein the bacterial strain is a Mycoplasma bacterial strain.

11. The composition of claim 1, wherein the Ig protease domain is from an Ig protease from Streptococcus krosus.

12. The composition of claim 1, wherein the Ig protease domain is of IdeS protease.

13. The composition of claim 1, wherein the Ig protease domain is of IdeZ protease.

14. The composition of claim 1, wherein the Ig protease domain is of IdeMC protease.

15. The composition of claim 1, wherein the Ig protease domain is of IdeSORK.

16. The composition of claim 1, wherein the Fc domain is a mouse Fc domain.

17-29. (canceled)

30. An Ig protease fusion protein comprising any one of the sequences provided herein.

31. A method of producing the Ig protease fusion protein of claim 1.

32. A method of administering any one of the Ig protease fusion proteins of claim 1 to a subject in need thereof, such as a subject that has an autoimmune disease, immunological disorder, GVHD or has had or will have a transplant.

33. A method of administering any one of the Ig protease fusion proteins of claim 1 to a subject in need thereof, such as a subject that is being or will be administered a therapeutic biologic.

34-41. (canceled)
